(19) United States
(12) Patent Application Publication
(10) Pub. No.: US 2025/0064798 A1
(43) Pub. Date: Feb. 27, 2025

(54) VILAZODONE COMPOSITION, PHARMACEUTICAL PREPARATION THEREOF, PREPARATION THEREFOR, AND USE THEREOF

(71) Applicant: SUNSHINE LAKE PHARMA CO., LTD., Dongguan, Guangdong (CN)

(72) Inventors: Lulu FAN, Dongguan (CN); Xin HUANG, Dongguan (CN); Jinsong YOU, Dongguan (CN); Manhua JIANG, Dongguan (CN); Fangfang HUANG, Dongguan (CN)

(73) Assignee: SUNSHINE LAKE PHARMA CO., LTD., Dongguan, Guangdong (CN)

(21) Appl. No.: 18/713,812

(22) PCT Filed: Nov. 30, 2022
(86) PCT No.: PCT/CN2022/135555

§ 371 Date: May 28, 2024

(30) Foreign Application Priority Data
Nov. 30, 2021 (CN) 202111443841.6

(51) Int. Cl. A61K 31/496; A61K 9/00; A61K 9/20

(52) U.S. Cl. CPC A61K 31/496; A61K 9/0053; A61K 9/2013; A61K 9/2018; A61K 9/2027; A61K 9/2054

(57) ABSTRACT

A vilazodone composition, a pharmaceutical preparation thereof, a preparation therefor, and a use thereof, relating to the pharmaceutical field. The composition contains an inclusion compound, the inclusion compound containing an active ingredient wrapped in an inclusion material; and the specification of the pharmaceutical preparation is 8 mg to 9 mg, 16 mg to 18 mg, or 32 mg to 36 mg. Under the condition that the specification of the pharmaceutical preparation can be reduced, the pharmacokinetics effect of the pharmaceutical preparation in a human body can still be equivalent to that of a reference listed drug, and the pharmaceutical preparation has an unexpected technical effect.

### FIELD OF THE INVENTION

The present invention relates to the field of pharmaceutical, and in particular to vilazodone composition and pharmaceutical preparation, preparation method and use thereof.

### BACKGROUND ART

Vilazodone is the first indole alkylamine antidepressant, belongs to selective serotonin reuptake inhibitor and 5-HT1A receptor partial agonist. The U.S. Food and Drug Administration approved vilazodone hydrochloride tablets (trade name: VIIBRYD®) for the treatment of major depressive disorder on Jan. 21, 2011. VIIBRYD® has serious food effects, the bioavailability is about 72% when taken with food, and the bioavailability is reduced by 50˜60% when taken on an empty stomach, which is difficult to meet the needs of clinical treatment. Therefore, the instructions of VIIBRYDR clearly state that the drug must be taken with food. Since the patients with depression are often accompanied by symptoms of loss of appetite, this method of administration, which must be taken with food, brings serious inconvenience to patients and leads to poor compliance, resulting in poor clinical treatment results.

In order to improve the bioavailability of vilazodone when taken on an empty stomach and weaken the influence of food effects, the patent applications CN 113164394A and CN 113164473 A used solid dispersion technology to increase the in vitro dissolution of vilazodone, and also increase the bioavailability of vilazodone under fasting conditions in animals.

Although the prior art has disclosed methods for increasing the dissolution of vilazodone, and the prepared low-specification (10 mg) vilazodone tablets also increased the bioavailability in animals, there are large species differences between animals and humans, animal data cannot directly reflect the absorption situation in the human body, let alone obtain dosage information for human medication. The inventors found through research that conducting pharmacokinetic tests in humans according to the existing technologies that can improve dissolution, improve bioavailability in animals, and eliminate food effects cannot achieve the technical effects of improving fasting bioavailability and eliminating food effects in human body.

During the clinical application of vilazodone, adverse reactions increase with the increase in dosage. At the same time, the marketed product must be taken with food, which brings inconvenience to patients. Therefore, developing a product that can be taken on an empty stomach or with food, and that can reduce the dosage of medication for patients and make it more convenient and safer for patients, is a difficult problem that the industry needs to solve urgently.

### SUMMARY

### Summary of the Invention

In order to solve the above problems, in the first aspect, the present invention provides a composition, which includes an inclusion complex, the inclusion complex comprises an active ingredient wrapped in an inclusion material, wherein the active ingredient is vilazodone or a pharmaceutically acceptable salt thereof; preferably, the pharmaceutically acceptable salt of vilazodone is a hydrochloride of vilazodone, that is, vilazodone hydrochloride, wherein, calculated based on vilazodone hydrochloride, the specification of the composition is 8 mg˜9 mg, 16 mg˜18 mg or 32 mg˜36 mg. The specification of vilazodone in the composition is smaller than that of commercial preparations, but it is bioequivalent to commercial preparations such as vilazodone hydrochloride tablets, and the oral preparations of vilazodone containing the composition can eliminate the food effect.

In the second aspect, the present invention provides a pharmaceutical preparation, which comprises the composition described in the first aspect and optionally at least one pharmaceutically acceptable adjuvant.

In the third aspect, the present invention provides a method for preparing the composition described in the first aspect.

In the fourth aspect, the present invention provides a method for preparing the pharmaceutical preparation described in the second aspect.

In the fifth aspect, the present invention provides use of the composition described in the first aspect.

### DETAILED DESCRIPTION OF THE INVENTION

The present invention provides a composition, pharmaceutical preparation, preparation method and use thereof.

In the first aspect, the present invention provides a composition.

The composition of the present invention comprises an inclusion complex, and the inclusion complex comprises an active ingredient wrapped in an inclusion material, wherein the active ingredient is vilazodone or a pharmaceutically acceptable salt thereof. In some embodiments, the pharmaceutically acceptable salt of vilazodone is a hydrochloride of vilazodone, that is, vilazodone hydrochloride; wherein, calculated based on vilazodone hydrochloride, the specification of the composition is 8 mg˜9 mg, 16 mg˜18 mg or 32 mg˜36 mg.

In some embodiments, the active ingredient is vilazodone or a pharmaceutically acceptable salt thereof other than vilazodone hydrochloride (i.e., non-vilazodone hydrochloride). When the active ingredient is vilazodone or other salts thereof (non-vilazodone hydrochloride), the specification of the composition can be calculated according to the specification of vilazodone hydrochloride to obtain the corresponding specification. In some embodiments, the active ingredient is vilazodone hydrochloride. In some embodiments, the active ingredient is a crystalline form of vilazodone. In some embodiments, the active ingredient is a crystalline form of vilazodone hydrochloride. In some embodiments, the active ingredient is an amorphous form of vilazodone hydrochloride. In some embodiments, the specification of the composition is 8 mg, 9 mg, 16 mg, 18 mg, 32 mg or 36 mg. In some embodiments, the specification of the composition is 8 mg, 16 mg or 32 mg. In some embodiments, the specification of the composition is 9 mg, 18 mg or 36 mg. In some embodiments, the specification of the composition is 8 mg. In some embodiments, the specification of the composition is 9 mg. In some embodiments, the specification of the composition is 16 mg. In some embodiments, the specification of the composition is 18 mg. In some embodiments, the specification of the composition is 32 mg. In some embodiments, the specification of the composition is 36 mg.

In some embodiments, an active ingredient molecule is contained within a cavity of an inclusion material molecule. In some embodiments, an active ingredient molecule is contained within the cavity of two inclusion material molecules.

In some embodiments, a composition comprises an inclusion complex, the inclusion complex comprises an active ingredient and an inclusion material, wherein the active ingredient is vilazodone or a pharmaceutically acceptable salt thereof, and wherein at least about 90% of the active ingredient is wrapped in the inclusion material; preferably, the pharmaceutically acceptable salt of vilazodone is vilazodone hydrochloride, wherein, calculated based on vilazodone hydrochloride, the specification of the composition is 8 mg˜9 mg, 16 mg˜18 mg or 32 mg˜36 mg. The high inclusion rate greatly improves the solubility and bioavailability of the drug under fasting condition or physiological condition of the small intestine.

In some embodiments, the inclusion rate of the inclusion complex of the present invention is at least about 90%, 95%, 98% or 99%.

In some embodiments, in the inclusion complex of the present invention, all the active ingredients are wrapped in the inclusion material, that is, the inclusion rate is 100%. Adopting this embodiment is helpful to eliminate the food effect, that is, it is helpful to make the pharmacokinetics in the human body equivalent between taking the medicine on an empty stomach and taking the medicine after eating.

The weight ratio of the active ingredient to the inclusion material of the present invention can be about 1:2.4 to 1:45.4. In some embodiments, the weight ratio of the active ingredient to the inclusion material is about 1:5 to 1:45.4. In some embodiments, the weight ratio of the active ingredient to the inclusion material is about 1:6.5 to 1:45.4. In some embodiments, the weight ratio of the active ingredient to the inclusion material is about 1:8 to 1:16.5.

The inclusion material of the present invention comprises cyclodextrin or derivatives thereof. In some embodiments, the inclusion material is selected from at least one of α-cyclodextrin, β-cyclodextrin, and γ-cyclodextrin and derivatives thereof. In some embodiments, the inclusion material comprises at least one of β-cyclodextrin and derivatives thereof. In some embodiments, the inclusion material comprises at least one of hydroxypropyl-β-cyclodextrin and sulfobutyl-β-cyclodextrin. In some embodiments, the inclusion material comprises at least one of hydroxypropyl-β-cyclodextrin sodium and sulfobutyl-β-cyclodextrin sodium.

The examples show that in the composition of the present invention, vilazodone is molecularly encapsulated in the cavity of cyclodextrin or its derivatives, which eliminates the influence of different crystalline forms due to different solubility, and has very high degree of dispersion; therefore, various crystalline forms of vilazodone or vilazodone hydrochloride may be used to form the compositions of the present invention. What is even more surprising is that the solubility of the inclusion complex of the present invention is obviously not affected by pH, thereby improving bioavailability and ensuring efficacy when administered on an empty stomach.

The composition of the present invention is in liquid or solid form. In some embodiments, the composition is in liquid form, such as an inclusion solution or suspension. In some embodiments, the composition is in solid form.

The preparation containing the composition of the present invention may be an oral preparation. In some embodiments, the preparation containing composition of the present invention is a tablet, a pill, a capsule, a granule, a soft capsule, a dry suspension or an oral liquid. In some embodiments, the preparation containing composition of the invention is tablet, pill, capsule, granule or soft capsule.

In some embodiments, the composition has a dissolution rate of about 85% or greater in a solution at pH 6-pH 7 for 15 minutes. In some embodiments, the composition has a dissolution rate of about 85% or greater in a solution at pH 6.8 for 15 minutes.

In some embodiments, the composition is administered orally; optionally, the percentage of AUC₀₋ₜ for administration in the fasting state and fed state is about 85% or greater when the composition is administered orally. In some embodiments, the percentage of AUC_{0-inf} for administration in the fasting state and fed state is about 85% or greater when the composition is administered orally.

In some embodiments, the composition is administered orally; optionally, when the composition is administered orally, the lower limit of the 90% confidence interval for the percentage of AUC₀₋ₜ for administration in the fed fasting state and the fasting fed state is about 80% or more, and the upper limit of the 90% confidence interval for the percentage of AUC₀₋ₜ for administration in the fed fasting state and the fasting fed state is about 125% or less. In some embodiments, when the composition is administered orally, the lower limit of the 90% confidence interval for the percentage of AUC_{0-inf} for administration in the fed fasting state and the fasting fed state is about 80% or more, and the upper limit of the 90% confidence interval for the percentage of AUC_{0-inf} for administration in the fed fasting state and the fasting fed state is about 125% or less.

In the second aspect, the present invention provides a pharmaceutical preparation.

A pharmaceutical preparation comprising the composition described in the first aspect.

The pharmaceutical preparation of the present invention also comprises at least one pharmaceutically acceptable adjuvants.

Calculated based on vilazodone hydrochloride, the specification of the pharmaceutical preparation of the present invention is 8 mg˜9 mg, 16 mg˜18 mg or 32 mg˜36 mg.

The pharmaceutically acceptable adjuvants of the present invention include at least one of fillers, disintegrants and lubricants. In some embodiments, pharmaceutically acceptable excipients comprise one or more intragranular excipients and one or more extragranular excipients.

In some embodiments, the inclusion material of the present invention comprises cyclodextrin or derivatives thereof. In some embodiments, the inclusion material of the present invention is selected from at least one of hydroxypropyl-β-cyclodextrin and sulfobutyl-β-cyclodextrin. In some embodiments, the inclusion material of the present invention is selected from at least one of hydroxypropyl-β-cyclodextrin sodium and sulfobutyl-β-cyclodextrin sodium.

In some embodiments, the weight ratio of the active ingredient of the present invention to the inclusion material is about 1:2.4-1:45.4, 1:5-1:45.4, 1:6.5-1:45.4 or 1:8-1:16.5.

In some embodiments, the specification of the pharmaceutical preparation of the present invention is 8 mg, 9 mg, 16 mg, 18 mg, 32 mg or 36 mg.

The pharmaceutical preparation of the present invention may be an oral preparation. In some embodiments, the pharmaceutical preparation is a tablet, a pill, a capsule, a granule, a soft capsule, a dry suspension or an oral liquid. In some embodiments, the pharmaceutical preparation is a tablet, a pill, a capsule, a granule or a soft capsule.

In some embodiments, in the pharmaceutical preparation, the active ingredient is vilazodone hydrochloride. In some embodiments, in the pharmaceutical preparation, the active ingredient is a crystalline form of vilazodone. In some embodiments, in the pharmaceutical preparation, the active ingredient is a crystalline form of vilazodone hydrochloride. In some embodiments, in the pharmaceutical preparation, the active ingredient is an amorphous form of vilazodone hydrochloride.

The fillers of the present invention comprise at least one of lactose, sucrose, fructose, fructo oligosaccharide, glucose, maltose, sugar powder, D-mannitol, erythritol, xylitol, corn starch, potato starch, rice starch, partial a starch, microcrystalline cellulose, calcium sulfate, calcium hydrogen phosphate and calcium carbonate.

The disintegrants of the present invention comprise at least one of starch, calcium carboxymethylcellulose, croscarmellose sodium, crospovidone, sodium carboxymethyl starch, hydroxypropyl methyl cellulose and low substituted hydroxypropyl cellulose.

The lubricants of the present invention comprise at least one of magnesium stearate, calcium stearate, sodium stearate fumarate, stearic acid, talc, polyethylene glycol, sucrose fatty acid ester, colloidal silica and micropowder silica gel.

In some embodiments, the pharmaceutically acceptable adjuvants or excipients of the present invention may also comprise a dissolution stabilizer, and the dissolution stabilizer is selected from at least one of hydroxypropyl methyl cellulose, hydroxypropyl cellulose, sodium carboxymethylcellulose, copolyvinyl, povidone, succinic acid, malic acid, citric acid anhydrous, citric acid monohydrate.

Based on the total weight of the preparation, the pharmaceutical preparation of the present invention comprises about 2.00% w/w to about 8.00% w/w active ingredient. In some embodiments, based on the total weight of the preparation, the pharmaceutical preparation comprises about 3.00% w/w to about 8.00% w/w active ingredient. In some embodiments, based on the total weight of the preparation, the pharmaceutical preparation comprises about 4.00% w/w to about 8.00% w/w active ingredient. In some embodiments, based on the total weight of the preparation, the pharmaceutical preparation comprises about 5.00% w/w to about 8.00% w/w active ingredient. In some embodiments, based on the total weight of the preparation, the pharmaceutical preparation comprises about 6.00% w/w to about 8.00% w/w active ingredient. In some embodiments, based on the total weight of the preparation, the pharmaceutical preparation comprises about 7.00% w/w to about 8.00% w/w active ingredient.

Based on the total weight of the preparation, the pharmaceutical preparation comprises about 20.00% w/w to about 75.00% w/w inclusion material. In some embodiments, based on the total weight of the preparation, the pharmaceutical preparation comprises about 60.00% w/w to about 75.00% w/w inclusion material. In some embodiments, based on the total weight of the preparation, the pharmaceutical preparation comprises about 60.00% w/w to about 72.00% w/w inclusion material. In some embodiments, based on the total weight of the preparation, the pharmaceutical preparation comprises about 60.23% w/w to about 71.43% w/w inclusion material.

Based on the total weight of the preparation, the pharmaceutical preparation of the present invention comprises about 20.00% w/w to about 80.00% w/w filler. In some embodiments, based on the total weight of the preparation, the pharmaceutical preparation comprises about 20.00% w/w to about 35.00% w/w filler. In some embodiments, based on the total weight of the preparation, the pharmaceutical preparation comprises about 22.00% w/w to about 32.00% w/w filler. In some embodiments, based on the total weight of the preparation, the pharmaceutical preparation comprises about 22.00% w/w to about 31.24% w/w filler.

Based on the total weight of the preparation, the pharmaceutical preparation of the present invention comprises about 0 to about 25.00% w/w disintegrant. In some embodiments, based on the total weight of the preparation, the pharmaceutical preparation comprises about 0% w/w to about 5.00% w/w disintegrant.

Based on the total weight of the preparation, the pharmaceutical preparation of the present invention comprises about 0 to about 2.00% w/w lubricant. In some embodiments, based on the total weight of the preparation, the pharmaceutical preparation comprises about 0.40% w/w to about 1.60% w/w lubricant. In some embodiments, based on the total weight of the preparation, the pharmaceutical preparation comprises about 0.48% w/w to about 1.54% w/w lubricant.

In some embodiments, based on the total weight of the preparation, the pharmaceutical preparation of the present invention comprises about 2.00% w/w to about 8.00% w/w vilazodone hydrochloride, about 60.00% w/w to about 72.00% w/w inclusion material, about 22.00% w/w to about 32.00% w/w filler, about 0% w/w to about 5.00% w/w disintegrant, and about 0.40% w/w to about 1.60% w/w lubricant.

In some embodiments, based on the total weight of the preparation, the pharmaceutical preparation of the present invention comprises about 7.00% w/w to about 8.00% w/w vilazodone hydrochloride, about 60.23% w/w to about 71.43% w/w inclusion material, about 22.00% w/w to about 31.24% w/w filler, about 0% w/w to about 5.00% w/w disintegrant, and about 0.48% w/w to about 1.54% w/w lubricant.

Based on the total weight of the preparation, the pharmaceutical preparation of the present invention comprises about 0-5.00% w/w dissolution stabilizer. In some embodiments, based on the total weight of the preparation, the pharmaceutical preparation of the present invention comprises about 0-4.00% w/w dissolution stabilizer.

In some embodiments, the dissolution rate of the active ingredient of the pharmaceutical preparation in a solution at pH 6-pH 7 for 15 minutes is 85% or more. In some embodiments, the dissolution rate of the active ingredient of the pharmaceutical preparation in a solution at pH 6.8 for 15 minutes is 85% or more.

In some embodiments, the pharmaceutical preparation is administered orally, and when the pharmaceutical formulation is administered orally, the percentage of AUC₀₋ₜ for administration in the fasting state and fed state may be about 85% or more. In some embodiments, the pharmaceutical preparation is administered orally, and when the pharmaceutical formulation is administered orally, the percentage of AUC_{0-inf} for administration in the fasting state and fed state may be about 85% or more.

In some embodiments, the pharmaceutical preparation is an oral preparation; optionally, when the pharmaceutical preparation is administered orally, the lower limit of the 90% confidence interval for the percentage of AUC₀₋ₜ for administration in the fed state and the fasting state is about 80% or more, and the upper limit of the 90% confidence interval for the percentage of AUC₀₋ₜ for administration in the fed state and the fasting state is about 125% or less. In some embodiments, when the pharmaceutical preparation is administered orally, the lower limit of the 90% confidence interval for the percentage of AUC_{0-inf} for administration in the fed state and the fasting state is about 80% or more, and the upper limit of the 90% confidence interval for the percentage of AUC_{0-inf} for administration in the fed state and the fasting state is about 125% or less.

In some embodiments, in the pharmaceutical preparation, all active ingredients are wrapped in inclusion materials, that is, the inclusion rate is 100%. The technical solution provided herein is beneficial to eliminating the food effect.

In the third aspect, the present invention provides a method for preparing the composition described in the first aspect.

A method for preparing the composition according to the first aspect comprising: dissolving an active ingredient and an inclusion material to form an inclusion solution.

In some embodiments, a method for the composition of the first aspect comprising: dissolving an active ingredient and an inclusion material to form an inclusion solution; and drying the inclusion solution to form a solid inclusion complex. In some embodiments, the method comprises dissolving an active ingredient and an inclusion material in a solvent (e.g., an aqueous solvent, such as water) to form an inclusion solution. In some embodiments, the method comprises dissolving an active ingredient and an inclusion material in a solvent (e.g., an aqueous solvent, such as water) for a period of time, such as at least about 1 hour, at least about 2 hours, at least about 4 hours to form an inclusion solution.

In some embodiments, the inclusion solution is prepared by mixing the active ingredient and inclusion material in an aqueous solvent (e.g., water). In some embodiments, the inclusion solution is prepared from a saturated aqueous solution with the active ingredient and inclusion material as solutes. In some embodiments, the inclusion complex is prepared by grinding. In some embodiments, the active ingredient, inclusion material and a sufficient amount of aqueous solvent are mixed and ground, for example, the active ingredient, inclusion material and water (e.g., per gram of active ingredient is mixed with at least about 5 ml, 10 ml or 20 ml of water) are ground for a sufficient time (e.g., at least about 1 hour, at least about 2 hours, at least about 4 hours) to form an inclusion solution. In some embodiments, the inclusion solution is prepared by an ultrasonic method. In some embodiments, a mixture of active ingredient and inclusion material in an aqueous solvent is sonicated until an inclusion solution is obtained.

In some embodiments, the method comprises increasing the temperature of the solution above room temperature and below the boiling point of the solvent, such as about 40° C.-100° C., about 60° C.-90° C., or about 80° C.

In some embodiments, the method comprises: (1) preparing an inclusion solution; and (2) optionally, drying the inclusion solution to obtain a solid inclusion complex.

In some embodiments, the solid inclusion complex is prepared by spray drying the inclusion solution. In some embodiments, the solid inclusion complex is prepared by freeze-drying the inclusion solution.

In some embodiments, the preparation method of the composition comprises: (1) preparing an inclusion solution, optionally, the method for preparing the inclusion solution can be selected from at least one of saturated aqueous solution, grinding method and ultrasonic method; and (2) obtaining a solid inclusion complex by spray drying or freeze-drying the inclusion solution.

In the fourth aspect, the present invention provides a method for preparing the pharmaceutical preparation described in the second aspect.

A method of preparing a pharmaceutical preparation according to the second aspect comprising mixing the composition according to the first aspect with at least one pharmaceutically acceptable adjuvant or excipient.

In some embodiments, a method for preparing the pharmaceutical preparation according to the second aspect, comprising: mixing the composition of the present invention with pharmaceutically acceptable adjuvants or excipients to obtain total mixed granules, wherein, the pharmaceutically acceptable adjuvants or excipients include at least one of fillers, disintegrants and lubricants; optionally, the total mixed granules are formed into a pharmaceutical preparation, for example, compressing into a tablet core.

In some embodiments, the method further comprises coating, for example, coating the tablet core.

In some embodiments, a method of preparing a pharmaceutical preparation according to the second aspect, comprising: a) dissolving the active ingredient and inclusion material to form an inclusion solution; b) adding intragranular excipients to the inclusion solution, granulating and drying to form dry granules; and c) mixing the extragranular excipients and dry granules to form a pharmaceutical preparation, such as compressing into a tablet core.

In some embodiments, the method further comprises coating, such as coating the tablet core.

In some embodiments, a method of preparing the pharmaceutical preparation according to the second aspect, comprising: a) dissolving the active ingredient and the inclusion material to form an inclusion solution; b) adding intragranular excipients such as fillers to the inclusion solution to form a mixture, then granulating and drying in a fluidized bed granulator to obtain dry granules; and c) mixing the dry granules with extragranular excipients to obtain total mixed granules, and forming the total mixed granules into a pharmaceutical preparation, such as tablet core, or filling the total mixed granules into a hard capsule.

In some embodiments, a method of preparing the pharmaceutical preparation according to the second aspect, comprising: a) dissolving the active ingredient and the inclusion material to form an inclusion solution; and b) adding a filler to the inclusion solution and drying to form dry granules; optionally, forming the dry granules into a pharmaceutical preparation.

In the composition or pharmaceutical preparation of the present invention, all active ingredients are wrapped in inclusion complex (i.e., the inclusion rate is 100%), and the composition or pharmaceutical preparation is beneficial to eliminating food effects, that is, it is beneficial to make the in vivo pharmacokinetics of administering the drug on an empty stomach equivalent to that of after eating.

In the fifth aspect, the present invention provides use of the composition described in the first aspect.

Use of the composition according to the first aspect in the manufacture of a medicament.

In some embodiments, a use of the composition according to the first aspect in the manufacture of a medicament for treating or preventing depression.

### DEFINITION OF TERMS

As used herein, the following words and phrases are generally intended to have the meanings stated below, unless the context indicates otherwise.

The term “AUC” refers to the area enclosed by the blood drug concentration curve versus the time axis.

The term “AUC₀₋ₜ” or “AUCₗₐₛₜ” refers to the area under the drug concentration-time curve from time 0 to the sample collection time t at which the last concentration accurately measured.

The term “AUC0-inf” or “AUC_{0-∞},” refers the area under the drug concentration-time curve from time 0 to infinite time (∞).

The term “Tₘₐₓ” refers to the time required to reach peak drug concentration after administration.

The term “Cₘₐₓ” refers to the maximum blood drug concentration after administration.

The term “comprise” and its variations, such as “include” or “contain” should be understood as an open expression, it means comprising the contents disclosed herein, but don't exclude other contents. When used to define compositions and methods, “basically consists of . . . ” or its grammatical variants shall indicate the exclusion of other elements of any importance to the composition and the method of preparation, but not factors that have no substantial effect on the composition and the method of preparation. “Consists of . . . ” or its grammatical variants shall indicate the exclusion of elements not explicitly enumerated. The embodiments defined by each of these transitional terms are within the scope of the present invention. For example, when a preparation is described as comprising components A, B, and C, the preparation is essentially composed of A, B, and C, and the preparation consists of A, B, and C, independently within the scope of the present invention.

The singular forms “a,” “an,” and “the” comprise the plurals unless the context clearly dictates otherwise. For example, “excipient” comprises a variety of excipients.

“Many” or “plurality” comprises plural references of two and more unless the context clearly dictates otherwise. For example, “many excipients” comprises two or more excipients.

As used herein, the term “about” in the context of a quantitative measurement refers to ±10%, ±5% or ±1% of the stated value. For example, “about 10” means 9-11, 9.5-10.5, or 9.9-10.1. The term “about X” also comprises “X”.

The description of the range of values in the text is intended to be used as a shorthand for the individual value that falls into the range. Each individual value is incorporated into this specification as if it is referenced separately herein unless otherwise stated herein.

The term “% w/w” as used herein refers to the mass/weight percent of a certain component in a composition or mixture containing that component. For example, if vilazodone is 50 mg present in a composition having a total weight of 100 mg, then vilazodone is present at a level of 50% w/w.

The term “pharmaceutically acceptable” refers to a material that is not biologically or otherwise undesirable, e.g., which material may be incorporated into a pharmaceutical preparation administered to a patient without causing any significant adverse biological effects or interacting in any deleterious manner with other components of the preparation. The preferably pharmaceutically acceptable carriers (e.g., carriers, adjuvants and/or other excipients) meet toxicological and manufacturing testing standards and/or inactive ingredients contained in the guidelines established by the US FDA.

The term “adjuvant” or “excipient”, or “pharmaceutically acceptable adjuvant” or “pharmaceutically acceptable excipient” refers to a filler, a diluent, a disintegrant, a precipitation inhibitor, a surfactant, a glidant, a binder, a lubricant and other excipients and carriers that are administered with the compound. The adjuvant or excipient described herein is described in “Remington's Pharmaceutical Sciences” by E. W. Martin.

The term “filler” refers to a compound that helps the drug form more easily. Non-limiting examples of filler include lactose, sucrose, fructose, fructo oligosaccharide, glucose, maltose, powdered sugar, D-mannitol, erythritol, xylitol and other sugar alcohols, corn starch, potato starch, rice starch, partial a starch and other starches, microcrystalline cellulose, calcium sulfate, calcium hydrogen phosphate, calcium carbonate and other inorganic salts.

The term “binder” refers to any pharmaceutically acceptable excipient that can be used to bind active and inert ingredients together to maintain a viscous and discrete portion. Non-limiting examples of binder include hydroxypropyl methyl cellulose, hydroxypropyl methylcellulose, povidone, copovidone and ethyl cellulose.

The term “disintegrant” refers to a substance added to a solid preparation to promote the fragmentation or disintegration of the preparation after administration and to permit release of the active ingredient. Non-limiting examples of disintegrant include starch, corn starch, sodium starch glycolate, croscarmellose sodium, crospovidone, modified corn starch, sodium carboxymethyl starch, povidone, pregelatin starch, calcium carboxymethylcellulose, low-substituted hydroxypropylcellulose or alginic acid.

The term “lubricant” refers to a substance added to a powder mixture to prevent the compacted powder material from sticking to equipment during tableting or encapsulation. Lubricant can help tablets eject from the mold and can improve powder flow. Non-limiting examples of lubricant include magnesium stearate, stearic acid, micronized silica gel, fat, calcium stearate, polyethylene glycol, sodium stearyl fumarate, sucrose fatty acid esters or talc; and solubilizers such as fatty acids, include lauric acid, oleic acid and C8/C10 fatty acids.

The term “coating” refers to a film on the surface of a substrate (such as a tablet). Coatings can be used to protect active ingredients from photodegradation. Non-limiting examples of coating materials include polyvinyl alcohol, hydroxyethyl cellulose, hydroxypropyl methyl cellulose, sodium carboxymethyl cellulose, polyethylene glycol 4000, and cellulose acetate phthalate.

Cyclodextrin and derivatives thereof are compounds that have sugar molecules (such as glucose) or derivatives thereof bound together in rings (cyclic oligosaccharides). Derivatives of cyclodextrin, including derivatives of α-cyclodextrin, β-cyclodextrin or γ-cyclodextrin, wherein one or more hydrogen atoms of the hydroxyl group are substituted by substituents, such as alkyl or substituted alkyl (—R), acyl (—C(O) R) and sulfate (—S(O)₂ OH or a salt thereof), where R is alkyl or substituted alkyl. In some embodiments, R is an alkyl group (C₁-C₆ alkyl) containing 1, 2, 3, 4, 5 or 6 carbon atoms, such as methyl, ethyl, propyl, isopropyl, butyl, isobutyl, pentyl, hexyl, etc. In some embodiments, R is an alkyl substituted with one or more substituents independently selected from hydroxyl and sulfate, such as hydroxymethyl (—CH₂OH), or sulfobutyl (—CH₂CH₂CH₂CH₂S(O)₂OH or salts thereof). Non-limiting examples of cyclodextrin and derivatives include α-cyclodextrin, β-cyclodextrin, γ-cyclodextrin, hydroxyethyl-β-cyclodextrin, hydroxypropyl-β-cyclodextrin (also known as hydroxypropyl cellulose β-cyclodextrin, β-cyclodextrin hydroxypropyl ether or HPBCD), 2-hydroxypropyl-β-cyclodextrin, sulfobutyl-β-cyclodextrin, dihydrogen-β-cyclodextrin, methyl-β-cyclodextrin, dimethyl-β-cyclodextrin, hydroxypropyl-γ-cyclodextrin, β-cyclodextrin-sulfobutyl ether sodium (also known as sulfobutyl ether-β-cyclodextrin, sulfobutyl ether-β-cyclodextrin sodium salt, sulfobutyl ether-β-cyclodextrin, sulfobutyl ether-β-cyclodextrin sodium salt), glucose cyclodextrin, maltodextrin, carboxymethyl cyclodextrin and sulfoalkyl cyclodextrin.

The “reference listed drug” or “RLD” mentioned in the present invention refers to the oral tablet approved by the US FDA, its number is NDA No. 022567, and sold with the brand name VIIBRYD®, which contains 10 mg, 20 mg or 40 mg of crystalline form IV vilazodone hydrochloride.

Vilazodone or pharmaceutically acceptable salt thereof refers to 5-(4-(4-(5-cyano-1H-indol-3-yl)butyl) piperazin-1-yl)benzofuran-2-carboxamide or a pharmaceutically acceptable salt thereof. Vilazodone hydrochloride refers to the monohydrochloride of vilazodone. The structural formula of vilazodone is shown below:

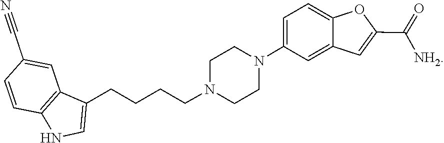 [SMILES: N#Cc1ccc2[nH]cc(CCCCN3CCN(c4ccc5oc(C(N)=O)cc5c4)CC3)c2c1]

### Beneficial Effects

Compared with the prior art, the composition or pharmaceutical preparation of the present invention comprises at least one of the following technical effects:

(1) Since the dosing method described in the instructions for the reference listed drug VIIBRYD® is to start with an initial dose of 10 mg once a day for 7 days, then 20 mg once a day for another 7 days, and then increase to 40 mg once a day. Different specifications of the reference listed drug VIIBRYD® are used in different clinical stages respectively. The bioequivalence of the product of the present invention and the reference listed drug is that 32 mg˜36 mg of the product of the present invention is bioequivalent to 40 mg of the reference listed drug VIIBRYD®, 16 mg˜18 mg of the product of the present invention is bioequivalent to 20 mg of the reference listed drug VIIBRYD®, and 8 mg˜9 mg of the product of the present invention is bioequivalent to 10 mg of the reference listed drug VIIBRYD®. The specification of the technical solution provided herein is 10%-20% lower than that of the reference listed drug, but it can still achieve the same in vivo pharmacokinetic effect as the reference listed drug, which has unexpected technical effects.

(2) In the technical solution provided herein, the drug can be taken on an empty stomach or with food, and both have good curative effects, greatly improve the patient's medication compliance, further reduce adverse reactions, and bring more clinical benefits to the patient.

### EXAMPLES

Those skilled in the art understand that the following examples are intended to illustrate the invention and are not to be construed as limiting the invention. Various modifications and variations of the present invention may occur to those skilled in the art. Unless otherwise specified, if specific techniques or conditions are not explicitly described in the following embodiments, those skilled in the art can proceed according to commonly used techniques or conditions in the field or according to product instructions. The drugs, reagents or instruments used herein are conventional products that are commercially available if the manufacturer is not indicated.

Wherein, the reference listed drug VIIBRYD® is vilazodone hydrochloride tablets developed by Forest Laboratory with specifications (calculated based on vilazodone hydrochloride) of 10 mg/tablet and 40 mg/tablet (calculated based on vilazodone hydrochloride). Unless otherwise specified, the following embodiments use the following detection methods to detect the content of vilazodone:

HPLC instrument model: Agilent 1260, chromatographic conditions: detection wavelength: UV 242 nm, chromatographic column: kromasil 100-5 C18 4.6 mm×150 mm, 5 μm, mobile phase: 0.02 M pH 6.0, dipotassium hydrogen phosphate and acetonitrile were 54:46 (V/V, volume ratio), flow rate: 1.0 mL/min, injection volume: 10 μL, running time: 4.5 min.

Unless otherwise specified, the following embodiments use the following dissolution method for dissolution:

Dissolution method: In the dissolution medium pH 6.8 that simulates intestinal conditions, a dissolution instrument was used to detect drug dissolution using the paddle method described in the United States Pharmacopoeia (i.e. USP II) at a rotation speed of 60 rpm. The solutions were taken at each dissolution time point, then filtered with a 0.45 μm microporous filter membrane, and the filtrate of each sample was analyzed by HPLC.

Unless otherwise specified, the term “Mean±SD” means mean±standard deviation; “min” means minutes; “M” means moles per liter; “V/V” means volume ratio; “pH” means acidity or alkalinity; “nm” means nanometers; “g” means grams; “ng” means nanograms; “mL” means milliliters; “h” means hours; “LC-MS-MS” means liquid chromatography-secondary mass spectrometry; “90% C.I.” means the 90% confidence interval; “n” or “N” in pharmacokinetic experiments means the number of subjects.

Unless otherwise specified, vilazodone hydrochloride in the following embodiments was crystal form XVI (crystal form XVI named in U.S. Pat. No. 8,673,921).

### Comparative Example 1 (Referring to the Most Preferred Solution of the Prior Art CN109922807A)

### (1) Preparation of Tablets

The coated tablets obtained from the prescription of Example 8 in the prior art CN109922807A have similar pharmacokinetic data in dogs under fasting and fed conditions compared with the coated tablets of other Examples in CN109922807A, that is, the coated tablets obtained from the prescription of Example 8 in CN109922807A have the best effect in eliminating food effects. Therefore, this prescription was used for comparative investigation herein.

The specific prescription composition of Comparative Example 1 is shown in Table 1. Part of vilazodone hydrochloride and sulfobutyl-β-cyclodextrin were added to purified water while stirring, and the mixture was stirred at 80° C. for 4 hours to obtain a clear inclusion solution. The inclusion solution was used as the granulation liquid for fluidized bed granulation, microcrystalline cellulose and lactose were used as granulation substrates, the mixture was subjected to fluidized bed granulation and dried to obtain dry granules, and then mixed with additional portion of vilazodone hydrochloride, crospovidone and sodium stearyl fumarate to form total mixed granules, which were compressed to obtain vilazodone hydrochloride tablets (specification: 10 mg (based on vilazodone hydrochloride)), and then coated.

TABLE 1
Tablet prescription composition of Comparative Example 1

| Composition | Amount (g) |
|---|---|
| Vilazodone hydrochloride | 53.90 |
| Sulfobutyl-β-cyclodextrin | 511.50 |
| Microcrystalline cellulose | 312.29 |
| Lactose | 80.74 |
| Vilazodone hydrochloride (additional) | 23.10 |
| Crospovidone (additional) | 107.69 |
| Sodium stearyl fumarate (additional) | 10.78 |

It can be seen from the above that in this prescription, part of the vilazodone hydrochloride is wrapped in the inclusion material (70% of the total amount of vilazodone hydrochloride), and part of the vilazodone hydrochloride is not wrapped (that is, the additional part, 30% of the total amount of vilazodone hydrochloride).

### (2) Dissolution Test

The above-mentioned coated tablets were subjected to an in vitro dissolution test in a dissolution medium of pH 6.8. The test results are shown in Table 2.

TABLE 2
Dissolution rate of the tablets of Comparative Example
1 in pH 6.8 dissolution medium (Mean ± SD)

| Experimental | | | | | |
|---|---|---|---|---|---|
| Examples | 10 min(%) | 15 min(%) | 20 min(%) | 30 m(%)in | 45 min(%) |
| Comparative | 70 ± 0.58 | 73 ± 0.58 | 74 ± 1.15 | 74 ± 0.58 | 75 ± 0.58 |
| Example 1 | | | | | |
| Reference | 2 ± 0.60 | 4 ± 0.60 | 4 ± 0.60 | 5 ± 0.00 | 6 ± 0.60 |
| listed drug | | | | | |
| (10 mg) | | | | | |

The dissolution results of this prescription are shown in Table 2. In pH 6.8 medium, the tablets of Comparative Example 1 significantly improve the dissolution of vilazodone hydrochloride. According to the cumulative dissolution calculation, in the fasting state, the dissolved amount of each tablet in the small intestine is about 7.5 mg.

### (3) Pharmacokinetic Tests in Animals

The coated tablet prepared in Example 1 was subjected to a crossover experiment under fasting and fed conditions in 3 Beagle dogs to verify the pharmacokinetic characteristics in vivo. Whole bloods were collected at 0.25h, 0.5h, 1 h, 2h, 4 h, 6h, 8 h, 10h, and 24h respectively to prepare plasma: 200˜400 μL of whole blood was taken and placed in a centrifuge tube, then K₂EDTA anticoagulant was added, the mixture was centrifuged within 60 minutes, then the plasma was taken and stored at −70° C., the content of vilazodone in the plasma was detected by LC-MS-MS, and the pharmacokinetic parameters were calculated by the non-compartmental model method of WinNonlin 6.3 software, the results are as shown in Table 3.

TABLE 3
Pharmacokinetic parameters of the prescription in Comparative
Example 1 in Beagle dogs under fasting and fed conditions

| | | | Cₘₐₓ | AUCₗₐₛₜ |
|---|---|---|---|---|
| Pharmacokinetic parameters | | Tₘₐₓ(h) | (ng/mL) | (h*ng/mL) |
| Fasting | Mean value | 0.67 | 111.04 | 382.91 |
| | Coefficient of variation % | 43.3 | 43.1 | 54.0 |
| Fed | Mean value | 2.0 | 96.52 | 382.64 |
| | Coefficient of variation % | 86.5 | 63.43 | 59.6 |

As shown in Table 3, the AUCₗₐₛₜ and Cₘₐₓ of the coated tablets of Comparative Example 1 in Beagle dogs are similar under fasting and fed conditions, which achieves bioequivalence and eliminates food effect. Since the in vivo pharmacokinetic data of the 10 mg-40 mg specifications are linear, under the same prescription, the in vivo pharmacokinetic data of the prescription with 40 mg specification can be derived from the in vivo pharmacokinetic data of the prescription with 10 mg specification. Therefore, the coated tablets of 10 mg specification of Comparative Example 1 eliminate the food effect in Beagle dogs, it can be derived that the coated tablets of 40 mg specification with the same prescription in Comparative Example 1 also eliminate the food effect in Beagle dogs (the same applies to other comparative examples or examples).

### (4) Clinical Trials

The coated tablets of Example 1 (specification: 10 mg (calculated based on vilazodone hydrochloride)) were used for clinical trials.

In order to confirm the pharmacokinetic characteristic of vilazodone hydrochloride tablets in the human body, the vilazodone hydrochloride tablets prepared in Comparative Example 1 were administered under fed and fasting states, and then evaluated the pharmacokinetic characteristic of commercial VIIBRYD® (10 mg) under eating.

For clinical trials, the criteria for evaluating whether to eliminate food effects refer to the FDA's BE guidance “Technical Guiding Principles for Food Effects on Bioavailability and Postprandial Bioequivalence Studies” regarding the elimination of food effects, that is, if the 90% confidence interval of the percentage of the pharmacokinetic data in the human body after administration in the fed state and fasting state is within the range of 80.00%-125.00%, it is considered that the food effect has been eliminated. The provisions for eliminating the food effect in other comparative examples and embodiments of the present invention are also stipulated in the same manner, and will not be described again.

This trial was a randomized, three-period crossover trial design conducted in India. This trial includes 18 healthy trial volunteers (18-45 years old (inclusive)) with body mass index (BMI) between 18.5-24.9 kg/m² (inclusive).

Fasting state: Supervised fasting for at least 10 hours before and 4 hours after dosing. Meals or snacks will be available 4 hours after dosing and at appropriate times thereafter. The meal plan was the same for all periods. No drinking water 1 hour before and 1 hour after administration, and taking the drug with 240 mL of water.

Fed state: Supervised fasting for at least 10 hours before eating a high-calorie, high-fat breakfast. 30 minutes before taking the drug, the subjects ate a high-calorie, high-fat breakfast. However, only subjects who consumed 800 calories or more were dosed. Meals or snacks will be available 4 hours after dosing and at appropriate times thereafter. The meal plan was the same for all periods. No drinking water 1 hour before and 1 hour after administration, and taking the drug with 240 mL of water.

Plasma sampling: During each of the 3 study phases, 24 blood samples were collected. Blood samples (1×3 mL) were collected at 0.0h within 1 hour before administration, and blood samples (1×3 mL) were collected at 0.5h, 1 h, 2h, 3 h, 3.5h, 4 h, 4.5h, 5 h, 5.5h, 6 h, 6.5h, 7 h, 8h, 12 h, 16h, 20 h, 24 h, 28h, 36h, 48h, 72h, 96h and 144h after administration. All samples were stored in vacuum containers containing K₂EDTA. The results are shown in Table 4.

TABLE 4
Pharmacokinetic results in human body of the prescription
in comparative example 1 and reference listed drug
Comparison of the pharmacokinetic results of the prescription
in Comparative Example 1 in human body under fed and fasting states

| Pharmacokinetic | T- Fed/ | 90% Confidence interval of |
|---|---|---|
| parameters | T-Fast ratio % | T-Fed/T-Fast ratio (%) |
| AUC₀₋ₜ (h*ng/mL) | 122.53 | 111.53-134.62 |
| AUC_{0-inf} (h*ng/mL) | 120.83 | 111.22-131.26 |
| Cₘₐₓ (ng/mL) | 123.79 | 111.74-137.21 |

Comparison of the pharmacokinetic results in human
body of the prescription (fed state) of Comparative
Example 1 and reference listed drug (fed state)

| Pharmacokinetic | T- Fed/ | 90% Confidence interval of |
|---|---|---|
| parameters | R-Fed ratio % | T-Fed/R-Fed ratio (%) |
| AUC₀₋ₜ (h*ng/mL) | 96.88 | 88.64-105.88 |
| AUC_{0-inf} (h*ng/mL) | 95.75 | 88.38-103.75 |
| Cₘₐₓ (ng/mL) | 92.49 | 83.21-102.81 |

Note: T-Fed represents the group in which the tablets described in Comparative Example 1 are administered in a fed state, T-Fast represents the group in which the tablets described in Comparative Example 1 are administered in a fasting state, and R-Fed represents the group in which the reference listed drug is administered in a fed state.

As shown in Table 4, when the vilazodone hydrochloride coated tablets prepared according to the prescription of Comparative Example 1 are administered in a fed state, the Cₘₐₓ and AUC_{0-inf} are approximately 123% and 120% of the results of administration in a fasting state, the 90% confidence intervals are 111.22%-131.26% and 111.74%-137.21% respectively, exceeding the range of 80.00-125.00%, and the food effect is not completely eliminated. The bioavailability of the coated tablets described in Comparative Example 1 under fasting state is about 20% lower than that of the fed state, and the coated tablets described in Comparative Example 1 are still at risk of insufficient efficacy when taken on an empty stomach.

It can be seen from the above results that the tablets described in Comparative Example 1 can completely eliminate the food effect in Beagle dogs, but it does not completely eliminate the food effect in human body, it can be seen that there are large species differences between animals and humans. Therefore, the absorption situation in animals cannot directly reflect the absorption situation in the human body, that is, it is difficult to confirm the equivalent dose in the human body through animal experiments, obtaining a lower and safer human dose requires a large number of experimental studies, which is not obvious.

### Comparative Example 2 (Referring to the Solution of the Prior Art CN113164473A)

### (1) Preparation of Solid Dispersion Tablets

According to the prescription proportions in Table 5, copovidone, poloxamer 188 and vilazodone hydrochloride powder were weighed into a beaker, then 60% acetone aqueous solution was added, the mixture was stirred to dissolve and placed in a Buchi small spray drying instrument (Inlet air temperature: 170° C., outlet air temperature: 90° C., rotation speed: 20 rpm), then sprayed drying to obtain spray-dried powder. The obtained powder was mixed evenly with the additional microcrystalline cellulose, crospovidone, micro-powder silica gel and magnesium stearate, the mixture was passed through a 40-mesh sieve and pressed into tablets according to the specification of 10 mg (calculated based on vilazodone hydrochloride) to prepare vilazodone hydrochloride tablets.

TABLE 5
Prescription composition of the solid dispersion
tablets in Comparative Example 2

| | Prescription | Preparation |
|---|---|---|
| Composition | proportion (%) | Process |
| Vilazodone hydrochloride | 5.00 | Spray drying |
| Copovidone | 30.00 | |
| Poloxamer 188 | 3.89 | |
| Microcrystalline cellulose | 50.11 | Mix |
| Crospovidone | 10.00 | |
| Micro-powder silica gel | 0.50 | |
| Magnesium stearate | 0.50 | |
| Total | 100.00 | Press |

### (2) Dissolution Test

The dissolution test was carried out according to the method of the present invention, and the results are shown in Table 6.

TABLE 6
Dissolution rate of Comparative Example 2
in pH 6.8 dissolution medium (Mean ± SD)

| Experimental | | | | | | |
|---|---|---|---|---|---|---|
| Examples | 5 min(%) | 10 min(%) | 15 min(%) | 20 min(%) | 30 min(%) | 45 min(%) |
| VIIBRYD ® | N/A | 2 ± 0.6 | 4 ± 0.6 | 4 ± 0.6 | 5 ± 0 | 6 ± 0.6 |
| (10 mg) | | | | | | |
| Comparative | 41 ± 3.06 | 85 ± 4.58 | 90 ± 1.53 | 92 ± 1.15 | 94 ± 1.53 | 94 ± 1.00 |
| Example 2 | | | | | | |

### (3) Pharmacokinetic Tests in Animals

The vilazodone hydrochloride tablets (10 mg specification (calculated based on vilazodone hydrochloride)) obtained in the above Comparative Example 2 were used to conduct an in vivo pharmacokinetic test in Beagle dogs. 6 Male, healthy adult Beagle dogs, with a weight range of 15-20 kg, were randomly divided into 2 groups, and a double-crossover and two-cycle test was conducted, the pharmacokinetic properties of the vilazodone tablets (10 mg specification (calculated based on vilazodone hydrochloride)) obtained in Comparative Example 2 were investigated respectively when administered on fasting and fed (high-fat, high-calorie food) states. After the animals were given oral administration, whole bloods were collected at 0.25h, 0.5h, 1 h, 2h, 4 h, 6h, 8 h, 10h, and 24h respectively; the above whole bloods were placed in a centrifuge tube containing K₂EDTA anticoagulant, centrifuged at 4° C. at 3500 rpm for 10 min, and then the upper plasma was collected and stored at −70° C. until detection.

The content of vilazodone in plasma was detected by LC-MS-MS method, and the pharmacokinetic parameters were calculated by WinNonlin 6.3 software non-compartmental model method. The food effect evaluation standard was calculated according to the Ratio value (ratio), for example, Fed/Fast Ratio can be calculated according to Ratio=Fed/Fast*100%. For a single animal in a given group of tested animals, the Ratio value of Cₘₐₓ and the Ratio value of AUC are calculated according to this formula, after the Ratio values of each animal are added, the sum is divided by the number of animals tested to obtain the average value. If the mean Ratio is in the range of about 70% to about 143% (due to the small number of tested animals and large variation, the 90% confidence interval is not statistically significant, therefore, for animal experiments, the 90% confidence interval is not compared, and only the Ratio value is evaluated using a custom range), it can be determined that the dosage form has no food effect and is equivalent when taken with food and on an empty stomach. That is to say, this dosage form has basically the same bioavailability and efficacy whether taken with food or on an empty stomach. The pharmacokinetic tests in animals of other comparative examples and examples below adopt the same evaluation method and will not be described again.

The experimental results are shown in Table 7, wherein Fast represents administration under fasting state, Fed represents administration under fed state; RLD is the reference listed drug VIIBRYD® (10 mg), and its pharmacokinetic data in animals are from patent application CN113164473A.

TABLE 7
Pharmacokinetic data of the tablets described
in Comparative Example 2 and reference listed
drug under fasting and fed states (Mean ± SD)

| | | Cₘₐₓ(ng/ | AUCₗₐₛₜ(h*ng/ |
|---|---|---|---|
| Group | Tₘₐₓ(h) | mL) | mL) |
| Comparative Example 2-Fed | 1.33 ± 0.58 | 56 ± 36 | 273 ± 170 |
| Comparative Example 2-Fast | 0.83 ± 0.29 | 62 ± 27 | 240 ± 120 |
| Comparative Example | — | 90.32 | 113.75 |
| 2-(Fed/Fast) % | | | |
| RLD-Fast | 1.50 ± 0.55 | 25 ± 19 | 109 ± 94 |
| RLD-Fed | 2.33 ± 1.86 | 77 ± 57 | 267 ± 188 |
| RLD-(Fed/Fast) % | — | 308.00 | 244.95 |

The results show that when the vilazodone hydrochloride tablets obtained in Comparative Example 2 are administered in fasting (Fast) and fed (Fed) states, Cₘₐₓ and AUCₗₐₛₜ are close, and the corresponding Ratio values are in the range of 80%-125%, indicating that the tablets of Comparative Example 2 can significantly improve the bioavailability in the fasting state, making the tablet bioequivalent in the fasting and fed states; while the Ratio values of Cₘₐₓ and AUCₗₐₛₜ of the commercially available preparation VIIBRYD® (10 mg) in fasting and fed states far exceed the range of 70%-143%, indicating a significant food effect.

It can be seen from the above results that the vilazodone hydrochloride tablets described in Comparative Example 2 prepared using solid dispersion technology can significantly improve the bioavailability of Beagle dogs in the fasting state. However, it is well known that solid dispersion is in a state of high energy and is susceptible to external interference and crystallization. The physiological environment in the human body is obviously more complex than that in the dogs, so the solid dispersion is more likely to be affected in the human body, and the larger the specification, the greater the impact.

### Comparative Example 3

### (1) Preparation of Solid Dispersion Tablets (10 mg)

According to the prescription proportions in Table 8, copovidone, hydroxypropyl methyl cellulose and vilazodone hydrochloride powder were weighed into a beaker, 60% acetone aqueous solution was added, the mixture was stirred to dissolve and placed in a Buchi small spray drying instrument (Inlet air temperature: 170° C., outlet air temperature: 90° C., rotation speed: 20 rpm), then sprayed drying to obtain spray-dried powder. The obtained powder was granulated with microcrystalline cellulose, magnesium aluminum silicate, crospovidone and micro-powder silica gel to obtain dry granules. The obtained dry granules were mixed evenly with the additional microcrystalline cellulose, crospovidone and magnesium stearate, the mixture was passed through a 40-mesh sieve and pressed into tablets according to the specification of 10 mg (calculated based on vilazodone hydrochloride), and coated with the coating liquid to prepare vilazodone hydrochloride tablets.

TABLE 8
Prescription composition of the solid dispersion
tablets in Comparative Example 3

| | Prescription | Preparation |
|---|---|---|
| Composition | proportion | Process |
| Vilazodone hydrochloride | 5.66 | Spray drying |
| Copovidone | 21.25 | |
| Hydroxypropyl methyl | 7.08 | |
| cellulose | | |
| Microcrystalline cellulose | 22.52 | Dry granulation |
| Magnesium aluminum silicate | 9.92 | |
| Crospovidone | 1.98 | |
| Micro-powder silica gel | 0.50 | |
| Microcrystalline cellulose | 22.66 | Mix |
| Crospovidone | 7.93 | |
| Magnesium stearate | 0.50 | |
| Total | 100.00 | Press |

### (2) Dissolution Test

The dissolution test was carried out according to the method of the present invention, and the results are shown in Table 9.

TABLE 9
Dissolution rate of Comparative Example 3
in pH 6.8 dissolution medium (Mean ± SD)

| Experimental | | | | | | |
|---|---|---|---|---|---|---|
| Examples | 5 min(%) | 10 min(%) | 15 min(%) | 20 min(%) | 30 min(%) | 45 min(%) |
| VIIBRYD ® | N/A | 2 ± 0.6 | 4 ± 0.6 | 4 ± 0.6 | 5 ± 0 | 6 ± 0.6 |
| (10 mg) | | | | | | |
| Comparative | 12 ± 1.53 | 38 ± 5.51 | 67 ± 4.04 | 76 ± 2.52 | 85 ± 1.00 | 86 ± 1.53 |
| Example 3 | | | | | | |
| (10 mg) | | | | | | |

### (3) Pharmacokinetic Tests in Animals

A crossover experiment was conducted on the coated tablets prepared in Example 3 (10 mg (calculated based on vilazodone hydrochloride)) in 6 Beagle dogs under fasting and fed states to verify the pharmacokinetic characteristics in the Beagle dogs. Whole bloods were collected at 0.25h, 0.5h, 1 h, 2h, 4 h, 6h, 8 h, 10h, and 24h respectively to prepare plasma: 200˜400 μL of whole blood was taken and placed in a centrifuge tube, K₂EDTA anticoagulant was added and the mixture was centrifuged within 60 minutes, then the plasma was taken and stored at −70° C. The content of vilazodone in the plasma was detected by LC-MS-MS, and the pharmacokinetic parameters were calculated by non-compartmental model method of WinNonlin 6.3 software, the results are shown in Table 10, wherein Fast represents administration on an empty stomach and Fed represents administration with food.

TABLE 10
Pharmacokinetic data of the tablets described in Comparative Example
3 in Beagle dogs under fasting and fed states (Mean ± SD)

| | | Cₘₐₓ(ng/ | AUCₗₐₛₜ(h*ng/ |
|---|---|---|---|
| Group | Tₘₐₓ(h) | mL) | mL) |
| Comparative Example 3-Fed | 4.58 ± 3.6 | 53.1 ± 26 | 375 ± 200 |
| Comparative Example 3-Fast | 1.5 ± 0.55 | 67 ± 32 | 364 ± 220 |

On the basis of Comparative Example 2, the inventors selected the better scheme of Comparative Example 3 for research, the bioavailability of the prepared tablets in animals increased significantly under fasting state, and the Cₘₐₓ was higher than that of fed state, the exposure amount was significantly higher than that of the reference listed drug under fasting and fed states (results are shown in Table 7). From this, it can be seen that the tablets described in this experiment can be of a lower specification, and it is generally speculated that the tablets will have the same effect in human body.

### (4) Clinical Trials

In order to verify the pharmacokinetic characteristics of the prescription of Comparative Example 3 in the human body, a clinical trial was conducted using vilazodone hydrochloride tablets (40 mg (based on vilazodone hydrochloride) of another specification re-prepared from the prescription of Comparative Example 3. The purpose is to investigate the food effect of the prescription of Comparative Example 3 under high specification conditions, and the bioequivalence of this high specification preparation and the reference listed drug VIIBRYD® (40 mg) under fed state.

This trial was a randomized, three-period crossover trial design conducted in India. This trial includes 30 healthy male adult volunteers, aged between 18-45 years old (inclusive), with body mass index (BMI) between 18.5-24.9 kg/m² (inclusive).

Fasting study: the volunteers were fasted for at least 10 hours overnight and taken the drug with 240±2 mL of water in the next morning.

Fed study: the volunteers were fasted for at least 10 hours overnight and had a high-fat and high-calorie breakfast in the next morning. After 30 minutes, the volunteers were taken the drug with 240±2 mL of water.

Plasma sampling: 25 blood samples were collected from each subject, each blood sample was 4.0 mL, plasma sample at 0.000h was collected before administration, and blood samples were collected at 0.5000h, 1.000h, 2.000h, 2.500h, 3.000h, 3.500h, 4.000h, 4.333h, 4.667h, 5.000h, 5.500h, 6.000h, 6.500h, 7.000h, 8.000h, 10.000h, 12.000h, 16.000h, 24.000h, 36.000h, 48.000h, 72.000h, 96.000h and 120.000h after administration, K₂EDTA was used as anticoagulant.

The test results are shown in Table 11.

TABLE 11
Pharmacokinetic results of the prescription of Comparative Example 3
(40 mg) in human body under fasting and fed states
Comparison of the pharmacokinetics results of the prescription of
Comparative Example 3 in humans under fed and fasting states

| Pharmacokinetic | T-Fed/T-Fast ratio | 90% Confidence interval of |
|---|---|---|
| parameters | (%) | T-Fed/T-Fast ratio (%) |
| AUC₀₋ₜ (h*ng/ml) | 210.7 | 174.7-254.0 |
| AUC_{0-inf} (h*ng/ml) | 207.9 | 173.2-249.4 |
| Cₘₐₓ (ng/ml) | 223.6 | 193.3-258.6 |

Comparison of the pharmacokinetics results of the prescription
of Comparative Example 3 under fasting state and the
reference listed drug under fed state in humans

| Pharmacokinetic | T-Fast/R-Fed ratio | 90% Confidence interval of |
|---|---|---|
| parameters | (%) | T-Fast/R-Fed ratio (%) |
| AUC₀₋ₜ (h*ng/ml) | 47.2 | 42.4-52.4 |
| AUC_{0-inf} (h*ng/ml) | 47.9 | 43.1-53.1 |
| Cₘₐₓ (ng/ml) | 44.1 | 38.6-50.4 |

Note: T-Fed represents the group in which the prescription of Comparative Example 3 (40 mg) is administered in a fed state, T-Fast represents the group in which the prescription of Comparative Example 3 (40 mg) is administered in a fasting state, and R-Fed represents the group in which the reference listed drug VIIBRYD® (40 mg) was administered in a fed state.

As shown in Table 11, the Cₘₐₓ and AUC_{0-inf} of the prescription of Comparative Example 3 (40 mg) in the fed state are approximately 2.2 times and 2.1 times that of the prescription in the fasting state respectively. The Cₘₐₓ and AUC_{0-inf} of the prescription in the fasting state are approximately 44% and 48% that of the reference listed drug in the fed state respectively, which indicates that this prescription has serious food effects in the human body.

In summary, the prescription of Comparative Example 3 can significantly improve the dissolution rate, and at the same time, the low specification tablets (10 mg) greatly improve the bioavailability of animals in the fasting state, which is significantly higher than the reference listed drug. However, the high specification tablets of this prescription have very low bioavailability under fasting state in the human body, even lower than that of the reference listed drug under fed state. It can be seen that pharmacokinetic data in humans cannot be derived from pharmacokinetic data in animals.

### Comparative Example 4 (Referring to the Solution of the Prior Art CN113164394A)

### (1) Preparation of Tablets

According to the prescription in Table 12, copovidone, poloxamer 188, vilazodone free base and fumaric acid were weighed, the mixture was mixed evenly and added to an extruder, and performed hot melt extrusion (extrusion temperature was 170° C., screw speed was 100 rpm), the extrudate was collected and crushed after cold cutting, and then passed through a 60-mesh sieve to obtain hot melt powder. The hot-melt powder was mixed evenly with the additional microcrystalline cellulose, crospovidone, and magnesium stearate. The obtained mixture was passed through a 40-mesh sieve, and compressed into 10 mg tablets (based on vilazodone free base) to prepare vilazodone tablets.

TABLE 12
Prescription composition of the solid dispersion tablets in
Comparative Example 4

| Composition | Prescription (g) | Preparation Process |
|---|---|---|
| Vilazodone free base | 10.00 | Hot melt powder |
| Copovidone | 60.00 | |
| Poloxamer 188 | 5.00 | |
| Fumaric acid | 4.00 | |
| Microcrystalline cellulose | 47.75 | Mix directly |
| Crospovidone | 22.50 | |
| Magnesium stearate | 0.75 | |
| Total | 150.00 | Press |

### (2) Pharmacokinetics Test in Animals

The vilazodone tablets (10 mg) prepared by the above prescription were used to conduct an in vivo pharmacokinetic test on Beagle dogs. 6 Male, healthy adult Beagle dogs, with a weight range of 15˜20 kg, were randomly divided into 2 groups and conducted a double-crossover and two-cycle test, the pharmacokinetic properties of self-developed vilazodone tablets (10 mg) under fasting and fed (high-fat, high-calorie food) states were investigated respectively. After oral administration to the animals, whole bloods were collected at 0.25h, 0.5h, 1 h, 2h, 4 h, 6h, 8 h, 10 h and 24h time points; the whole bloods were placed in a centrifuge tube containing K₂EDTA anticoagulant, then centrifuged at 3500 rpm for 10 minutes at 4° C., the upper plasma was collected and stored at −70° C. until detection. The content of vilazodone in plasma was detected by LC-MS-MS method, and the pharmacokinetic parameters were calculated by non-compartmental model method of WinNonlin 6.3 software.

The specific results are shown in Table 13, wherein Fast represents administration under fasting state, Fed represents administration under fed state; RLD is the reference listed drug VIIBRYD® (10 mg (calculated based on vilazodone hydrochloride), and the animal PK data are from Patent application CN113164394A.

TABLE 13
In vivo pharmacokinetic data of Comparative Example 4
prescription (10 mg) in dogs under fasting and fed states (Mean)

| Group | Cₘₐₓ (ng/mL) | AUCₗₐₛₜ (h*ng/ml) |
|---|---|---|
| Comparative Example 4-Fast | 84 | 276 |
| Comparative Example 4-Fed | 66 | 220 |
| Comparative Example 4-(Fed/Fast) | 78.57% | 79.71% |
| RLD-Fast | 25 | 109 |
| RLD-Fed | 77 | 267 |
| RLD-(Fed/Fast) | 308.00% | 244.95% |

As shown in Table 13, the low specification (10 mg) vilazodone tablets of the prescription in Comparative Example 4 can significantly improve the bioavailability in the fasting state, while The Cₘₐₓ and AUCₗₐₛₜ ratios of commercially available preparation VIIBRYD® (10 mg) under fed and fasting states far exceed the range of 70%-143%, which indicates a significant food effect and very low bioavailability under fasting state.

In summary, the currently disclosed solid dispersion technologies (CN113164394A, CN113164473A) significantly improve the dissolution rate of vilazodone in vitro, and the prepared low specification solid dispersion tablets also eliminate the food effect in animals. As we all know, solid dispersions are in a high energy unstable state, for poorly soluble drugs, they are more likely to precipitate and crystallize under high specification conditions, resulting in reduced dissolution rate and bioavailability. The physiological environment of the human body is more complex than that of animals, solid dispersions are more affected. Therefore, the pharmacokinetic characteristics of low specification solid dispersion tablets in the body may not be able to accurately predict the absorption under high specification conditions, and it is even more difficult to predict when the species is different.

The currently disclosed inclusion technology (patent CN109922807A) also significantly improves the in vitro dissolution rate of vilazodone, when reducing the effective dissolution dose in the fasting state, it achieves the effect of eliminating the effects of food in animals, but the food effects are only partially eliminated in humans, and pharmacokinetic results in humans do not reproduce those in animals.

Commercially available vilazodone hydrochloride tablets (trade name: VIIBRYD®) have serious food effects and must be taken with food to achieve clinical therapeutic effects, this method of administration brings serious inconvenience to patients with depression who suffer from loss of appetite, and the adverse reactions of vilazodone are linearly related to the dose. Therefore, the invention of an oral preparation of vilazodone that can eliminate the food effect and still achieve bioequivalence to the reference listed drug in the human body even though the specification is 10%-20% lower than the reference listed drug, it has great significance for the clinical treatment of patients with depression.

### Example 1

### (1) Preparation of Tablets

According to the prescription in Table 14, the prescribed amount of purified water was weighed and heated to 80° C.-90° C., then sulfobutyl-β-cyclodextrin sodium was added into the water to dissolve under stirring, and then vilazodone hydrochloride was added into the solution, the mixture was stirred to dissolve and included, the inclusion liquid was used as the granulation liquid, microcrystalline cellulose and colloidal silica were used as substrate, the mixture was performed fluidized bed granulation and dried to obtain dry granules, and then mixed with the lubricant sodium stearyl fumarate and tableted to prepare tablet cores with a specification of 40 mg (calculated based on vilazodone hydrochloride). The tablet cores were coated with the coating liquid to prepare coated tablets containing the vilazodone hydrochloride inclusion complex.

TABLE 14
Prescription composition of Example 1

| Composition | Unit dose (mg) | Ratio (%) |
|---|---|---|
| Vilazodone hydrochloride | 40.00 | 7.14 |
| Sulfobutyl-β-cyclodextrin sodium | 400.00 | 71.43 |
| Microcrystalline cellulose | 109.00 | 19.46 |
| Colloidal silica | 3.00 | 0.54 |
| Sodium stearyl fumarate | 8.00 | 1.43 |
| Total | 560.00 | 100.00 |

### (2) Dissolution Test

The dissolution test was carried out according to the method of the present invention, and the results are shown in Table 15.

TABLE 15
Dissolution data of the prescription of Example
1 in pH 6.8 dissolution medium (Mean ± SD)

| Experimental | | | | | | |
|---|---|---|---|---|---|---|
| Examples | 5 min(%) | 10 min(%) | 15 min(%) | 20 min(%) | 30 min(%) | 45 min(%) |
| VIIBRYD ® | 3 ± 0.58 | 3 ± 0.58 | 3 ± 0.58 | 3 ± 0.58 | 3 ± 0.00 | 3 ± 0.00 |
| (40 mg) | | | | | | |
| Example 1 | 46 ± 10.50 | 83 ± 10.60 | 96 ± 2.89 | 99 ± 0.58 | 98 ± 0.58 | 98 ± 0.58 |

As shown in Table 15, the dissolution rate of the coated tablet (40 mg) obtained in Example 1 in the pH 6.8 medium is close to 100%, while the dissolution rate of the reference listed drug under the same conditions is 3%, which is extremely low.

### (3) Clinical Trials

In order to verify the pharmacokinetic characteristics of the coated tablets of Example 1 in the human body, clinical trials were conducted using the vilazodone hydrochloride inclusion complex-coated tablets prepared in Example 1.

The highest specification of commercially available vilazodone hydrochloride tablets is 40 mg, for poorly soluble drugs, the higher the specification, the lower the dissolution rate, that is, the more obvious the food effect. Therefore, high specification preparations were used to conduct clinical trials to verify the pharmacokinetics of the composition or pharmaceutical preparation provided by the present invention and the reference listed drug in the human body.

This trial is a randomized, two-period crossover trial design conducted in India, with the purpose of investigating the food effect of the composition or pharmaceutical preparation provided by the present invention. This trial includes 28 healthy male adult volunteers, aged between 18-45 years old (inclusive), with body mass index (BMI) between 18.5-24.9 kg/m² (inclusive).

Fasting study: the volunteers were fasted for at least 10 hours overnight and taken the drug with 240 ml of water in the next morning.

Fed study: the volunteers were fasted for at least 10 hours overnight, had a high-fat and high-calorie breakfast in the next morning, and taken the drug with 240 mL of water 30 minutes later.

Plasma sampling: 22 blood samples were collected from each subject, each blood sample was 4 mL. Plasma sample was collected at 0.000h before administration, and blood samples were collected at 0.500h, 1.000h, 2.000h, 3.000h, 3.500h, 4.000h, 4.500h, 5.000h, 5.500h, 6.000h, 6.500h, 7.000h, 9.000h, 12.000h, 16.000h, 24.000h, 36.000h, 48.000h, 72.000h, 96.000h and 144.000h after administration, K₂EDTA was used as an anticoagulant. Results are as shown in Table 16.

TABLE 16
Pharmacokinetic results of the prescription in Example 1 and reference
listed drugs in human body under fasting and fed states

| | Geometric least | | | 90% Confidence |
|---|---|---|---|---|
| Pharmacokinetic | squares mean | | Ratio of | interval of Ratio |
| parameters | T2 (Fed) | T1 (Fast) | T2/T1 (%) | of T2/T1 (%) |
| Cₘₐₓ (ng/mL) | 109.211 | 116.570 | 93.7 | 85.12-103.12 |
| AUC₀₋ₜ (ng · h/mL) | 1946.229 | 1904.288 | 102.2 | 93.48-111.74 |
| AUC_{0-inf} (ng · h/mL) | 1992.168 | 1946.972 | 102.3 | 93.68-111.76 |
| | | | | 90% Confidence |
| Pharmacokinetic | | | Ratio of | interval of Ratio |
| parameters | R2 (Fed) | R1 (Fast) | R2/R1 (%) | of R2/R1 (%) |
| Cₘₐₓ (ng/mL) | 88.282 | 24.728 | 357.0 | 211.42-602.86 |
| AUC₀₋ₜ (ng · h/mL) | 1496.233 | 500.557 | 298.9 | 185.27-482.28 |
| AUC_{0-inf} (ng · h/mL) | 1532.818 | 527.752 | 290.4 | 184.76-456.57 |

Note: T2 (Fed) represents the group in which the coated tablets obtained in Example 1 are administered in a fed state, T1 (Fast) represents the group in which the coated tablets obtained in Example 1 are administered in a fasting state, R2 (Fed) represents the group in which the reference listed drug (VIIBRYD®, 40 mg) is administered in a fed state, and R1 (Fast) represents the group in which the reference listed drug (VIIBRYD®, 40 mg) is administered in a fasting state.

As shown in Table 16, in the human body, the Cₘₐₓ and AUC_{0-inf} of the coated tablet obtained in Example 1 administered in the fed state were 93.7% and 102.3% of the results of the coated tablet administered in the fasting state, respectively. The Cₘₐₓ and AUC_{0-inf} of the reference listed drug (VIIBRYDR, 40 mg) administered in the fed state were 357.0% and 290.4% of the results of the reference listed drug (VIIBRYDR, 40 mg) administered in the fasting state, respectively. It can be seen that in the human body, the reference listed drug has serious food effects, but the coated tablets described in Example 1 can completely eliminate the food effects and have significant beneficial effects. The data shows that the in vivo exposure of the coated tablet obtained in Example 1 is increased, and its AUC_{0-inf} administered in the fasting state is 27% higher than the AUC_{0-inf} of the reference listed drug administered in the fed state.

### Example 2

### (1) Preparation of Tablets

According to the prescription in Table 17, vilazodone hydrochloride and sulfobutyl-β-cyclodextrin sodium were added into the prescribed amount of purified water while stirring, and the mixture was stirred at 80° C.-90° C. until a clear inclusion solution was obtained. The inclusion solution was used as the granulation liquid for fluidized bed granulation, microcrystalline cellulose and colloidal silica were used as substrates, and the mixture was performed fluidized bed granulation to obtain granules. The obtained granules were fluidized bed dried to form dry granules. The dry granules were mixed with additional sodium stearyl fumarate to form mixed granules, and the mixed granules were compressed into tablets to prepare vilazodone hydrochloride inclusion tablet cores with a specification of 40 mg (calculated based on vilazodone hydrochloride), then coated with the coating liquid to prepare coated tablets containing the vilazodone hydrochloride inclusion complex.

TABLE 17
Prescription composition of Example 2

| Composition | Unit dose (mg) | Ratio (%) |
|---|---|---|
| Vilazodone hydrochloride | 40.00 | 7.69 |
| Sulfobutyl-β-cyclodextrin sodium | 360.00 | 69.23 |
| Microcrystalline cellulose | 109.00 | 20.96 |
| Colloidal silica | 3.00 | 0.58 |
| Sodium stearyl fumarate | 8.00 | 1.54 |
| Total | 520.00 | 100.00 |

### (2) Dissolution Test

The above-mentioned coated tablets were subjected to an in vitro dissolution test in a dissolution medium of pH 6.8. The test results are shown in Table 18.

TABLE 18
Dissolution data of the prescription of Example
2 in pH 6.8 dissolution medium (Mean ± SD)

| Experimental | | | | | | |
|---|---|---|---|---|---|---|
| Examples | 5 min(%) | 10 min(%) | 15 min(%) | 20 min(%) | 30 min(%) | 45 min(%) |
| VIIBRYD ® | 4 ± 0.55 | 4 ± 0.75 | 4 ± 0.52 | 4 ± 0.41 | 4 ± 0.00 | 4 ± 0.52 |
| (40 mg) | | | | | | |
| Example 2 | 31 ± 5.51 | 67 ± 8.54 | 88 ± 6.56 | 96 ± 2.31 | 96 ± 0.58 | 96 ± 0.00 |

The dissolution results of this prescription are shown in Table 18. After slightly reducing the dosage of sulfobutyl-β-cyclodextrin sodium in the preparation provided by the present invention (compared to Example 1), the dissolution of the prepared tablets in pH 6.8 medium is still close to 100%

### (3) Clinical Trials

Clinical trials were conducted using the coated tablets prepared according to the prescription of Example 2. This trial was a randomized, three-period, three-crossover trial design conducted in India. The purpose is to investigate the food effect of the composition or pharmaceutical preparation provided by the present invention and its bioequivalence with commercially available products. The trial included 30 healthy male adult volunteers, aged between 18-45 years old (inclusive), with a body mass index (BMI) between 18.5-24.9 kg/m² (inclusive).

Fasting study: the volunteers were fasted for at least 10 hours overnight and taken the drug with 240±2 mL of water in the next morning.

Fed study: the volunteers were fasted for at least 10 hours overnight and had a high-fat and high-calorie breakfast in the next morning. After 30 minutes, the volunteers were taken the drug with 240±2 mL of water.

Plasma sampling: 25 blood samples were collected from each subject, each blood sample was 4.0 mL. Plasma samples were collected at 0.000h before administration, and blood samples were collected at 0.500h, 1.000h, 2.000h, 2.500h, 3.000h, 3.500h, 4.000h, 4.333h, 4.667h, 5.000h, 5.500h, 6.000h, 6.500h, 7.000h, 8.000h, 10.000h, 12.000h, 16.000h, 24.000h, 36.000h, 48.000h, 72.000h, 96.000h and 120.000h after administration, K₂EDTA was used as an anticoagulant. The test results are shown in Table 19.

TABLE 19
Pharmacokinetic results of the prescription in Example 2 and reference
listed drugs in human body under fasting and fed states

| | T-Fed/R-Fed | |
|---|---|---|
| Pharmacokinetic | Ratio (%) of | 90% C.I (%) of the Ratio |
| parameters | T-Fed/R-Fed | of T-Fed/R-Fed |
| Cₘₐₓ (ng/ml) | 116.82 | 104.60-130.65 |
| AUC₀₋ₜ (ng · h/ml) | 110.11 | 102.38-118.38 |
| AUC_{0-∞} (ng · h/ml) | 110.07 | 102.46-118.24 |
| | T-Fed/T-Fast Food effect | |
| Pharmacokinetic | Ratio (%) of | 90% C.I (%) of the Ratio |
| parameters | T-Fed/T-Fast | of T-Fed/T-Fast |
| Cₘₐₓ (ng/ml) | 88.3 | 83.33~93.54 |
| AUC₀₋ₜ (ng · h/ml) | 100.7 | 95.02~106.69 |
| AUC_{0-∞} (ng · h/ml) | 100.6 | 95.07~106.47 |

Note: T-Fed represents the group of the salt-coated tablets obtained in Example 2 that are administered in a fed state, T-Fast represents the group of the coated tablets obtained in Example 2 that are administered in a fasting state, and R-Fed represents the group of the reference listed drug (VIIBRYD®, 40 mg) is administered in a fed state.

As shown in Table 19, the Cₘₐₓ and AUC_{0-∞}, of the coated tablets obtained in Example 2 when administered in a fed state are 88.3% and 100.6% that of the coated tablets administered in a fasting state, and are 116.82% and 110.07% that of the results of the reference listed drug administered in a fed state. It can be seen that the coated tablet obtained in Example 2 completely eliminates the food effect, and compared with the results of the reference listed drug administered in the fed state, the bioavailability of the coated tablet when administered in the fasting state is increased by about 10%.

### Example 3

### (1) Preparation of Tablets

According to the prescription in Table 20, under the heating conditions of the prescribed amount of purified water, vilazodone hydrochloride and sulfobutyl-β-cyclodextrin sodium were added into the purified water, the mixture was stirred at 80° C. until an inclusion solution was obtained. The inclusion solution was used as the granulation liquid for fluidized bed granulation, microcrystalline cellulose and colloidal silica were used as substrates, and the mixture was performed fluidized bed granulation to obtain granules. The obtained granules were fluidized bed dried to form dry granules. The dry granules were mixed with additional sodium stearyl fumarate to form mixed granules, and the mixed granules were compressed into tablets to prepare vilazodone hydrochloride tablet cores with a specification of 36 mg (calculated based on vilazodone hydrochloride). Coating powder was stirred with purified water to prepare a coating suspension, and the 36 mg specification (calculated based on vilazodone hydrochloride) vilazodone hydrochloride tablet cores were coated to prepare vilazodone hydrochloride coated tablet with a specification of 36 mg (calculated based on vilazodone hydrochloride). The coated tablet was subjected to an in vitro dissolution test in a dissolution medium of pH 6.8. The dissolution results are shown in Table 23.

TABLE 20
Prescription composition of Example 3

| Composition | Unit dose (mg) | Ratio (%) |
|---|---|---|
| Vilazodone hydrochloride | 36.00 | 7.50 |
| Sulfobutyl-β-cyclodextrin sodium | 324.00 | 67.50 |
| Microcrystalline cellulose | 115.20 | 24.00 |
| Colloidal silica | 2.40 | 0.50 |
| Sodium stearyl fumarate | 2.40 | 0.50 |
| Total | 480.00 | 100.00 |

### Example 4

### (1) Preparation of Tablets

According to the prescription in Table 21, under the heating conditions of the prescribed amount of purified water, vilazodone hydrochloride and sulfobutyl-β-cyclodextrin sodium were added into the purified water, and the mixture was stirred at 80° C. until a clear inclusion solution was obtained. The inclusion solution was used as the granulation liquid for fluidized bed granulation, microcrystalline cellulose and colloidal silica were used as substrates, and the mixture was performed fluidized bed granulation to obtain granules. The obtained granules were fluidized bed dried to form dry granules. The dry granules were mixed with additional sodium stearyl fumarate and sodium carboxymethyl starch to form mixed granules, and the mixed granules were compressed into tablets to prepare vilazodone hydrochloride tablet cores with a specification of 18 mg (calculated based on vilazodone hydrochloride). Then the tablet cores were coated with a coating solution to prepare vilazodone hydrochloride coated tablets with a specification of 18 mg (calculated based on vilazodone hydrochloride). The coated tablet was subjected to an in vitro dissolution test in a dissolution medium of pH 6.8. The dissolution results are shown in Table 23.

TABLE 21
Prescription composition of Example 4

| Composition | Unit dose (mg) | Ratio (%) |
|---|---|---|
| Vilazodone hydrochloride | 18.00 | 7.14 |
| Sulfobutyl-β-cyclodextrin sodium | 162.00 | 64.28 |
| Microcrystalline cellulose | 57.60 | 22.86 |
| Colloidal silica | 1.20 | 0.48 |
| Sodium carboxymethyl starch | 12.00 | 4.76 |
| Sodium stearyl fumarate | 1.20 | 0.48 |
| Total | 252.00 | 100.00 |

### Example 5

### (1) Preparation of Tablets

According to the prescription in Table 22, under the heating conditions of the prescribed amount of purified water, vilazodone hydrochloride and sulfobutyl-β-cyclodextrin sodium were added into the purified water, the mixture was stirred at 80° C. until an inclusion solution was obtained. The inclusion solution was used as the granulation liquid for fluidized bed granulation, microcrystalline cellulose and colloidal silica were used as substrates, and the mixture was performed fluidized bed granulation to obtain granules. The obtained granules were fluidized bed dried to form dry granules. The dry granules were mixed with additional sodium stearyl fumarate and crospovidone to form mixed granules, and the mixed granules were compressed into tablets to prepare vilazodone hydrochloride tablet cores with a specification of 9 mg (calculated based on vilazodone hydrochloride), then the tablet cores were coated with a coating solution to prepare vilazodone hydrochloride coated tablets with a specification of 9 mg (calculated based on vilazodone hydrochloride. The coated tablet was subjected to an in vitro dissolution test in a dissolution medium of pH 6.8. The dissolution results are shown in Table 23.

TABLE 22
Prescription composition of Example 5

| Composition | Unit dose (mg) | Ratio (%) |
|---|---|---|
| Vilazodone hydrochloride | 9.00 | 7.14 |
| Sulfobutyl-β-cyclodextrin sodium | 81.00 | 64.28 |
| Microcrystalline cellulose | 28.80 | 22.86 |
| Colloidal silica | 0.60 | 0.48 |
| Crospovidone | 6.00 | 4.76 |
| Sodium stearyl fumarate | 0.60 | 0.48 |
| Total | 126.00 | 100.00 |

### (2) Dissolution Test

The coated tablets described in Example 3, Example 4 and Example 5 were respectively subjected to in vitro dissolution tests in a dissolution medium of pH 6.8. The dissolution results are shown in Table 23.

TABLE 23
Dissolution data of vilazodone hydrochloride coated tablets
of Examples 3, 4 and 5 in pH 6.8 medium (Mean ± SD)

| Experimental | | | | | | |
|---|---|---|---|---|---|---|
| Examples | 5 min(%) | 10 min(%) | 15 min(%) | 20 min(%) | 30 min(%) | 45 min(%) |
| Example 3 | 50 ± 6.02 | 90 ± 4.40 | 97 ± 0.89 | 97 ± 0.52 | 97 ± 0.75 | 97 ± 0.63 |
| Example 4 | 45 ± 3.65 | 88 ± 3.27 | 99 ± 1.56 | 99 ± 1.07 | 99 ± 1.27 | 99 ± 0.97 |
| Example 5 | 56 ± 4.51 | 94 ± 2.89 | 98 ± 1.00 | 99 ± 1.00 | 99 ± 1.53 | 99 ± 1.00 |

As shown in Table 23, compared with the reference listed drug, the vilazodone coated tablets with a specification of 36 mg, 18 mg and 9 mg described in Example 3, Example 4 and Example 5 have lower specifications respectively, and the dissolution rates of the three specifications of coated tablets in the pH 6.8 medium are close to 100%.

### Example 6

### (1) Preparation of Tablets

According to the prescription in Table 24, vilazodone hydrochloride and sulfobutyl-β-cyclodextrin sodium were added into a purified water while stirring, the mixture was stirred at 80° C. until a clear inclusion solution was obtained. The inclusion solution was granulated and dried through a fluidized bed to obtain vilazodone hydrochloride inclusion granules. The vilazodone hydrochloride inclusion granules and sodium stearyl fumarate were mixed to obtain total mixed granules, then pressed into tablets and coated with a coating liquid to obtain coated tablets with a specification of 32 mg (calculated based on vilazodone hydrochloride). The coated tablets were subjected to an in vitro dissolution test in a dissolution medium of pH 6.8. The dissolution results are shown in Table 27.

TABLE 24
Prescription composition of Example 6

| Composition | Unit dose (mg) | Ratio (%) |
|---|---|---|
| Vilazodone hydrochloride | 32.00 | 7.53 |
| Sulfobutyl-β-cyclodextrin sodium | 256.00 | 60.23 |
| Microcrystalline cellulose | 132.76 | 31.24 |
| Colloidal silica | 2.12 | 0.50 |
| Sodium stearyl fumarate | 2.12 | 0.50 |
| Total | 425.00 | 100.00 |

### Example 7

### (1) Preparation of Tablets

According to the prescription in Table 25, vilazodone hydrochloride and sulfobutyl-β-cyclodextrin sodium were added into a purified water while stirring, the mixture was stirred at 80° C. until a clear inclusion solution was obtained. The inclusion solution was granulated and dried through a fluidized bed to obtain vilazodone hydrochloride inclusion granules. The vilazodone hydrochloride inclusion granules were mixed with sodium carboxymethyl starch and sodium stearyl fumarate to obtain total mixed granules, and then pressed into tablets, and coated with a coating liquid to obtain coated tablets with a specification of 16 mg (calculated based on vilazodone hydrochloride). The coated tablets were subjected to an in vitro dissolution test in a dissolution medium of pH 6.8. The dissolution results are shown in Table 27.

TABLE 25
Prescription composition of Example 7

| Composition | Unit dose (mg) | Ratio (%) |
|---|---|---|
| Vilazodone hydrochloride | 16.00 | 8.00 |
| Sulfobutyl-β-cyclodextrin sodium | 128.00 | 64.00 |
| Microcrystalline cellulose | 44.00 | 22.00 |
| Colloidal silica | 1.00 | 0.50 |
| Sodium carboxymethyl starch | 10.00 | 5.00 |
| Sodium stearyl fumarate | 1.00 | 0.50 |
| Total | 200.00 | 100.00 |

### Example 8

### (1) Preparation of Tablets

According to the prescription in Table 26, vilazodone hydrochloride and sulfobutyl-β-cyclodextrin sodium were added into a purified water while stirring, the mixture was stirred at 80° C. until a clear inclusion solution was obtained. The inclusion solution was granulated and dried through a fluidized bed to obtain vilazodone hydrochloride inclusion granules. The vilazodone hydrochloride inclusion granules were mixed with crospovidone and sodium stearyl fumarate to obtain total mixed granules, then pressed into tablets and coated with a coating solution to obtain coated tablets with a specification of 8 mg (calculated based on vilazodone hydrochloride). The coated tablets were subjected to an in vitro dissolution test in a dissolution medium of pH 6.8. The dissolution results are shown in Table 27.

TABLE 26
Prescription composition of Example 8

| Composition | Unit dose (mg) | Ratio (%) |
|---|---|---|
| Vilazodone hydrochloride | 8.00 | 8.00 |
| Sulfobutyl-β-cyclodextrin sodium | 64.00 | 64.00 |
| Microcrystalline cellulose | 22.00 | 22.00 |
| Colloidal silica | 0.50 | 0.50 |
| Crospovidone | 5.00 | 5.00 |
| Sodium stearyl fumarate | 0.50 | 0.50 |
| Total | 100.00 | 100.00 |

### (2) Dissolution Test

The coated tablets described in Example 6, Example 7 and Example 8 were respectively subjected to in vitro dissolution tests in a dissolution medium of pH 6.8. The dissolution results are shown in Table 27.

TABLE 27
Dissolution data of vilazodone hydrochloride coated
tablets in pH 6.8 medium of Examples 6-8 (Mean ± SD)

| Experimental | | | | | | |
|---|---|---|---|---|---|---|
| Examples | 5 min(%) | 10 min(%) | 15 min(%) | 20 min(%) | 30 min(%) | 45 min(%) |
| Example 6 | 58 ± 1.26 | 90 ± 0.98 | 96 ± 0.52 | 96 ± 0.55 | 95 ± 0.52 | 96 ± 0.52 |
| Example 7 | 57 ± 4.62 | 87 ± 5.62 | 95 ± 1.94 | 96 ± 1.03 | 97 ± 0.75 | 97 ± 1.47 |
| Example 8 | 77 ± 7.49 | 95 ± 1.72 | 96 ± 1.47 | 96 ± 1.21 | 97 ± 1.72 | 98 ± 1.38 |

As shown in Table 27, compared with the reference listed drug, the low specification vilazodone coated tablets (specifications are 32 mg, 16 mg and 8 mg respectively) described in Example 6, Example 7 and Example 8 have respectively reduced specifications, and the dissolution rates of the three specifications in pH 6.8 medium are close to 100%.

### Example 9—Example 11

### (1) Preparation of Tablets

According to the prescription in Table 28, the prescribed amount of hydroxypropyl-β-cyclodextrin sodium was weighed and added into purified water, the mixture was stirred to dissolve, then heated to 85° C., and then vilazodone hydrochloride API was added, and the resulting solution was stirred until complete inclusion to obtain a clear inclusion solution. The heating was turned off, hydroxypropyl methyl cellulose and citric acid monohydrate were added to the mixture, then stirred to dissolve to obtain a clear composition solution. The drug-containing solution was put in a fluidized bed granulator, and sprayed on the base material (microcrystalline cellulose+micronized silica gel), granulated to obtain dry granules; then prescribed proportion of sodium stearyl fumarate was added and the mixture was mixed evenly, and pressed into tablets according to the specifications of 36 mg, 18 mg, and 9 mg respectively to obtain vilazodone hydrochloride tablets with a specification of 36 mg, 18 mg, and 9 mg respectively.

TABLE 28
The prescription composition of vilazodone hydrochloride tablets of
Examples 9-11

| | Example 9 | Example 10 | Example 11 | |
|---|---|---|---|---|
| | Unit dose | Unit dose | Unit dose | Ratio |
| Composition | (mg) | (mg) | (mg) | (%) |
| Vilazodone hydrochloride | 36.00 | 18.00 | 9.00 | 7.20 |
| Hydroxypropyl-β- | 324.00 | 162.00 | 81.00 | 64.80 |
| cyclodextrin sodium | | | | |
| Hydroxypropyl methyl | 5.00 | 2.50 | 1.25 | 1.00 |
| cellulose | | | | |
| Citric acid monohydrate | 15.00 | 7.50 | 3.75 | 3.00 |
| Microcrystalline cellulose | 112.50 | 56.25 | 28.13 | 22.50 |
| Micro-powder silica gel | 2.50 | 1.25 | 0.63 | 0.50 |
| Sodium stearyl fumarate | 5.00 | 2.50 | 1.25 | 1.00 |
| Total | 500.00 | 250.00 | 125.00 | 100.00 |

### (2) Dissolution Test

The coated tablets described in Example 9, Example 10 and Example 11 were respectively subjected to in vitro dissolution tests in a dissolution medium of pH 6.8. The dissolution results are shown in Table 29.

TABLE 29
Dissolution data of the vilazodone hydrochloride tablets
in pH 6.8 medium of Examples 9-11 (Mean ± SD)

| Experimental | | | | | | |
|---|---|---|---|---|---|---|
| Examples | 5 min(%) | 10 min(%) | 15 min(%) | 20 min(%) | 30 min(%) | 45 min(%) |
| Example 9 | 33 ± 5.89 | 72 ± 9.83 | 90 ± 6.95 | 97 ± 1.72 | 98 ± 0.84 | 98 ± 1.47 |
| Example 10 | 53 ± 5.24 | 86 ± 2.8 | 98 ± 1.03 | 99 ± 0.82 | 99 ± 1.03 | 100 ± 1.21 |
| Example 11 | 63 ± 3.67 | 92 ± 2.66 | 96 ± 1.38 | 95 ± 1.37 | 96 ± 0.75 | 97 ± 0.75 |

As shown in Table 29, compared with the reference listed drug, the vilazodone hydrochloride tablets described in Examples 9-11 prepared using hydroxypropyl-β-cyclodextrin sodium have lower specifications, and the in vitro dissolution rates of these three specifications of vilazodone hydrochloride tablets in pH 6.8 medium have been significantly improved, and the dissolution rates are close to 100%.

### Example 12: Clinical Trial

The vilazodone hydrochloride coated tablets with a specification of 36 mg (calculated based on vilazodone hydrochloride) prepared in Example 3 were used for clinical trials. To verify the food effect of the vilazodone hydrochloride coated tablets with a specification of 36 mg (calculated based on vilazodone hydrochloride) under fed or fasted states, and its bioequivalence with commercial VIIBRYD® (40 mg) under fed state.

This trial was a randomized, three-period crossover trial design conducted in India. This trial includes 30 healthy male adult volunteers, aged between 18-45 years old (inclusive), with body mass index (BMI) between 18.5-24.9 kg/m² (inclusive).

Fasting study: the volunteers were fasted for at least 10 hours overnight and taken the drug with 240±2 mL of water in the next morning.

Fed study: the volunteers were fasted for at least 10 hours overnight and had a high-fat and high-calorie breakfast in the next morning. After 30 minutes, the volunteers were taken the drug with 240±2 mL of water.

Plasma sampling: 25 blood samples were collected from each subject, each blood sample was 4 mL. Plasma sample was collected at 0.000h before administration, and blood samples were collected at 0.500h, 1.000h, 2.000h, 2.500h, 3.000h, 3.500h, 4.000h, 4.333h, 4.667h, 5.000h, 5.500h, 6.000h, 6.500h, 7.000h, 8.000h, 10.000h, 12.000h, 16.000h, 24.000h, 36.000h, 48.000h, 72.000h, 96.000h and 120.000h after administration, K₂EDTA was used as an anticoagulant.

The test results are shown in Table 30.

TABLE 30
Pharmacokinetic parameters of the prescription in Example 3
in human body under fasting and fed states

| | T-Fed/R-Fed | |
|---|---|---|
| Pharmacokinetic | Ratio of T-Fed/R-Fed | 90% Confidence interval of |
| parameters | (%) | Ratio of T-Fed/R-Fed (%) |
| Cₘₐₓ (ng/ml) | 105.18 | 92.96-119.01 |
| AUC₀₋ₜ (ng · h/ml) | 98.28 | 90.55-106.55 |
| AUC_{0-∞} (ng · h/ml) | 98.17 | 90.56-106.34 |
| | T-Fed/T-Fast Food effect | |
| Pharmacokinetic | Ratio of T-Fed/T-Fast | 90% Confidence interval of |
| parameters | (%) | Ratio of T-Fed/T-Fast (%) |
| Cₘₐₓ (ng/ml) | 88.3 | 83.33-93.54 |
| AUC₀₋ₜ (ng · h/ml) | 100.7 | 95.02-106.69 |
| AUC_{0-∞} (ng · h/ml) | 100.6 | 95.07-106.47 |

Note: T-Fed represents the group of coated tablets in Example 3 are administered in fed state, T-Fast represents the group of coated tablets in Example 3 are administered in fasting state, and R-Fed represents the group of reference listed drug (40 mg) is administered in fed state.

As shown in Table 30, the 90% confidence interval of the Ratio of Cₘₐₓ in the fed and fasting states of the vilazodone hydrochloride coated tablets with 36 mg (calculated based on vilazodone hydrochloride) prepared in Example 3 of the present invention, the 90% confidence intervals of the Ratio of AUC_{0-∞}, in the fed and fasting states, and the 90% confidence intervals of the Ratio of AUC₀₋ₜ in the fed and fasting states are all between 80%-125%, and the 90% confidence interval of the Ratio between the Cₘₐₓ of the coated tablet described in Example 3 in the fed state and the Cₘₐₓ of the reference listed drug (VIIBRYDR, 40 mg) in the fed state, the 90% confidence interval of the Ratio between the AUC₀₋ₜ of the coated tablet described in Example 3 in the fed state and the AUC₀₋ₜ of the reference listed drug (VIIBRYDR, 40 mg) in the fed state, the 90% confidence interval of the Ratio between the AUC_{0-∞}, of the coated tablet described in Example 3 in the fed state and the AUC_{0-∞}, of the reference listed drug (VIIBRYD®, 40 mg) in the fed state are all between 80%-125%. It can be seen that the coated tablet described in Example 3 completely eliminates the food effect in the human body, and the bioavailability in the fed state is similar to the result of the reference listed drug in the fed state.

Since the in vivo pharmacokinetic data of different specifications of this product are linear, under the same prescription, the in vivo pharmacokinetic data of the prescriptions with 18 mg and 9 mg specifications can be deduced from the in vivo pharmacokinetic data of the prescription with 36 mg specification of the present invention. Since the product with 36 mg specification of the present invention is bioequivalent to the reference listed drug VIIBRYD® with 40 mg specification, it can be deduced that 18 mg product of the present invention is bioequivalent to 20 mg reference listed drug VIIBRYD®, and 9 mg product of the present invention is bioequivalent to 10 mg reference listed drug VIIBRYD®.

The method of the present invention has been described through preferred embodiments. Related person can clearly realize and apply the techniques disclosed herein by making some changes, appropriate alterations or combinations to the methods without departing from spirit, principles and scope of the present disclosure. Skilled in the art can learn from this article to properly improve the process parameters to implement the preparation method. Of particular note is that all similar substitutions and modifications to the skilled person is obvious, and they are deemed to be included in the present invention.

1. A composition comprising an inclusion complex, the inclusion complex comprising an active ingredient wrapped in an inclusion material, wherein the active ingredient is vilazodone hydrochloride, calculated based on vilazodone hydrochloride, the specification of the composition is 8 mg˜9 mg, 16 mg˜18 mg or 32 mg˜36 mg.

2. The composition of claim 1, wherein the inclusion material comprises cyclodextrin or derivatives thereof; optionally, the inclusion material comprises at least one of hydroxypropyl-β-cyclodextrin and sulfobutyl-β-cyclodextrin;
optionally, the inclusion material comprises at least one of hydroxypropyl-β-cyclodextrin sodium and sulfobutyl-β-cyclodextrin sodium.

3. The composition of claim 1, wherein the weight ratio of the active ingredient to the inclusion material is about 1:2.4-1:45.4, 1:5-1:45.4, 1:6.5-1:45.4 or 1:8-1:16.5.

4. The composition of claim 1, the specification of the composition is 8 mg, 9 mg, 16 mg, 18 mg, 32 mg or 36 mg.

5. The composition of claim 1, the dissolution rate of the composition in a solution at pH 6-pH 7 for 15 minutes is about 85% or more; or the dissolution rate of the composition in a solution at pH 6.8 for 15 minutes is about 85% or more.

6. The composition of claim 1, the composition is administered orally; optionally, when the composition is administered orally, the percentage of AUC_{0-inf} for administration in the fasting state and fed state is about 85% or more; or when the composition is administered orally, the percentage of AUC_{0-inf} for administration in the fasting state and fed state is about 85% or more.

7. The composition of claim 6, the composition is administered orally; optionally, when the composition is administered orally, the lower limit of the 90% confidence interval for the percentage of AUC₀₋ₜ for administration in the fed state and the fasting state is about 80% or more, and the upper limit of the 90% confidence interval for the percentage of AUC₀₋ₜ for administration in the fed state and the fasting state is about 125% or less; or when the composition is administered orally, the lower limit of the 90% confidence interval for the percentage of AUC_{0-inf} for administration in the fed state and the fasting state is about 80% or more, and the upper limit of the 90% confidence interval for the percentage of AUC_{0-inf} for administration in the fed state and the fasting state is about 125% or less.

8. A pharmaceutical preparation comprising the composition of claim 1 and optionally at least one pharmaceutically acceptable adjuvants or excipients.

9. The pharmaceutical preparation of claim 8, wherein the pharmaceutically acceptable adjuvants or excipients include at least one of fillers, disintegrants and lubricants.

10. The pharmaceutical preparation of claim 9, wherein the fillers comprise at least one of lactose, sucrose, fructose, fructo oligosaccharide, glucose, maltose, sugar powder, D-mannitol, erythritol, xylitol, corn starch, potato starch, rice starch, partial a starch, microcrystalline cellulose, calcium sulfate, calcium hydrogen phosphate and calcium carbonate;
optionally, the disintegrants comprise at least one of starch, calcium carboxymethylcellulose, croscarmellose sodium, crospovidone, sodium carboxymethyl starch and low substituted hydroxypropyl cellulose;
optionally, the lubricants comprise at least one of magnesium stearate, calcium stearate, sodium stearate fumarate, stearic acid, talc, polyethylene glycol, sucrose fatty acid ester, colloidal silica and micropowder silica gel.

11. The pharmaceutical preparation of claim 8, wherein, based on the total weight of the pharmaceutical preparation, the content of the active ingredient is about 2.00% w/w˜8.00% w/w;
optionally, based on the total weight of the pharmaceutical preparation, the content of the inclusion material is about 20.00% w/w˜75.00% w/w;
optionally, based on the total weight of the pharmaceutical preparation, the content of the filler is about 20.00% w/w˜80.00% w/w;
optionally, based on the total weight of the pharmaceutical preparation, the content of the disintegrant is about 0˜25.00% w/w;
optionally, based on the total weight of the pharmaceutical preparation, the content of the lubricant is about 0˜2.00% w/w.

12. The pharmaceutical preparation of claim 8, the dissolution rate of the pharmaceutical preparation in a solution at pH 6-pH 7 for 15 minutes is about 85% or more; the dissolution rate of the pharmaceutical preparation in a solution at pH 6.8 for 15 minutes is about 85% or more.

13. The pharmaceutical preparation of claim 8, the pharmaceutical preparation is an oral preparation; optionally, when the pharmaceutical preparation is administered orally, the percentage of AUC₀₋ₜ for administration in the fasting state and fed state is about 85% or more; or when the pharmaceutical preparation is administered orally, the percentage of AUC_{0-inf} for administration in the fasting state and fed state is about 85% or more.

14. The pharmaceutical preparation of claim 8, the pharmaceutical preparation is an oral preparation; optionally, when the pharmaceutical preparation is administered orally, the lower limit of the 90% confidence interval for the percentage of AUC₀₋ₜ for administration in the fed state and the fasting state is about 80% or more, and the upper limit of the 90% confidence interval for the percentage of AUC₀₋ₜ for administration in the fed state and the fasting state is about 125% or less; or when the pharmaceutical preparation is administered orally, the lower limit of the 90% confidence interval for the percentage of AUC_{0-inf} for administration in the fed state and the fasting state is about 80% or more, and the upper limit of the 90% confidence interval for the percentage of AUC_{0-inf} for administration in the fed state and the fasting state is about 125% or less.

15. A method for preparing the composition of claim 1 comprising: dissolving an active ingredient and an inclusion material to form an inclusion solution.

16. The method of claim 15, the method further comprising: drying the inclusion solution to form a solid inclusion complex.

17. A method for preparing the pharmaceutical preparation of claim 8 comprising: mixing a composition and one or more adjuvants or excipients, wherein the composition comprising an inclusion complex, the inclusion complex comprising an active ingredient wrapped in an inclusion material, wherein the active ingredient is vilazodone hydrochloride, calculated based on vilazodone hydrochloride, the specification of the composition is 8 mg˜9 mg, 16 mg˜18 mg or 32 mg˜36 mg.

18. A method of treating or preventing depression in a subject comprising administering to the subject a therapeutically effective amount of the composition of claim 1.
